(19) United States
(12) Patent Application Publication
(10) Pub. No.: US 2025/0120643 A1
(43) Pub. Date: Apr. 17, 2025

(54) INGESTIBLE AND/OR IMPLANTABLE ARTICLES FOR MONITORING ELECTRICAL SIGNALS AND RELATED METHODS

(71) Applicants: Massachusetts Institute of Technology, Cambridge, MA (US); The Brigham and Women's Hospital, Inc., Boston, MA (US)

(72) Inventors: Robert S. Langer, Newton, MA (US); Carlo Giovanni Traverso, Newton, MA (US); Siheng You, Somerville, MA (US); So-Yoon Yang, Cambridge, MA (US); Adam Gierlach, Toronto (CA); George Selsing, Jamaica Plain, MA (US)

(73) Assignees: Massachusetts Institute of Technology, Cambridge, MA (US); The Brigham and Women's Hospital, Inc., Boston, MA (US)

(21) Appl. No.: 18/913,061

(22) Filed: Oct. 11, 2024

Related U.S. Application Data
(60) Provisional application No. 63/589,401, filed on Oct. 11, 2023.

(51) Int. Cl. A61B 5/392; A61B 5/00; A61B 5/296

(52) U.S. Cl. CPC A61B 5/392; A61B 5/0004; A61B 5/0031; A61B 5/296; A61B 5/4238; A61B 5/686; A61B 5/6861; A61B 5/7246; A61B 2562/0209; A61B 2562/043; A61B 2562/164

(57) ABSTRACT

Some aspects of the present disclosure are related to articles for measuring electrical signals internal to a subject, e.g., of the gastrointestinal tract (GI tract). The articles, in some embodiments, are ingestible and/or implantable. In some embodiments, the articles comprise a substrate comprising a plurality of electrodes, the substrate having a Young's elastic modulus of greater than or equal to 0.01 MPa and less than or equal to 200 MPa. In some such cases, the Young's elastic modulus of the substrate facilitates elastic recoil of the substrate such that at least a portion of the plurality of electrodes contact a surface of a tissue at the location internal to the subject (e.g., the GI tract) and thus facilitate measurement of electrical signals at the location internal to the subject. Other aspects are related to methods of using the articles, for example, to monitor electrical signals from the location internal to the subject.

### TECHNICAL FIELD

Ingestible and/or implantable articles for monitoring electrical signals and related methods are generally described.

### BACKGROUND

Measuring electrical signals internal to the body can provide information about the functioning of various organs and/or systems within the body. For example, electrical activity associated with the GI tract (e.g., from neurons associated therewith) regulates motility and hormone secretion, and dysfunction or other irregularities with such signals may indicate various GI disorders, such as gastroparesis. However, measuring such electronic signals within the body is challenging due to the depth within the body from which the signals originate. Accordingly, improved systems and methods for measuring electrical signals internal to the body are needed.

### SUMMARY

Ingestible and/or implantable articles for monitoring electrical signals and related methods are generally described. The subject matter of the present disclosure involves, in some cases, interrelated products, alternative solutions to a particular problem, and/or a plurality of different uses of one or more systems and/or articles.

Some aspects disclosed herein relate to articles. In some embodiments, the article is for monitoring electrical signals at a location internal to a subject. In some embodiments, the article for monitoring electrical signals at a location internal to a subject comprises a substrate comprising a plurality of electrodes, the article sized and adapted for ingestion and/or implantation at the location internal to the subject, wherein the substrate has a Young's elastic modulus of greater than or equal to 0.01 MPa and less than or equal to 200 MPa, and wherein at the location internal to the subject, the substrate undergoes elastic recoil sufficient such that at least a portion of the plurality of electrodes contact a surface of a tissue at the location internal to the subject.

Some aspects disclosed herein relate to methods. In some embodiments, the method is for monitoring electrical signals at a location internal to a subject using an article. In some embodiments, the method for monitoring electrical signals at a location internal to a subject using an article comprises contacting a surface of a tissue with at least a portion of a plurality of electrodes of the article at the location internal to the subject and using the at least a portion of the plurality of electrodes to determine an electrical signal that is correlated with a gastric slow wave and/or gastric motility.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIGS. 1A-1B are schematic diagrams of exemplary articles, according to some embodiments;

FIGS. 2A-2D show exemplary articles and how exemplary uses of such articles, according to some embodiments;

FIGS. 3A-3E show articles and evaluation of such articles for use in vivo according to some embodiments;

FIG. 4 show articles, according to some embodiments;

FIG. 5 shows gastric clearance of articles, according to some embodiments;

FIG. 6 shows implantation of articles, according to some embodiments;

FIGS. 7A-7D show measurements attained using an article, according to some embodiments;

FIG. 8 is a schematic diagram showing the configuration of an article, according to some embodiments;

FIGS. 9A-9E shows EKG and respiration signals measured using an article, according to some embodiments;

FIGS. 10A-10D shows measurements using an article after prokinetic delivery during animal activities, according to some embodiments;

FIG. 11 shows measurements using an article prior to delivery of azithromycin, according to some embodiments;

FIG. 12 shows power changes measured in various channels of the article after azithromycin dosing, according to some embodiments;

FIGS. 13A-13B show slow wave power and frequency during a free moving experiment, according to some embodiments;

FIG. 14 shows long period gastric electrical activity using an article, according to some embodiments;

FIG. 15 shows impedance between electrodes of an article, according to some embodiments;

FIG. 16 is a schematic diagram of the printed circuit board of an article, according to some embodiments;

FIGS. 17A-17B are images of custom flexible electrodes, according to some embodiments;

FIGS. 18A-D are measurement results from a commercial acquisition system, according to some embodiments;

FIGS. 19A-19B show a schematic and image of an article, according to some embodiments;

FIGS. 20A-20C show electronics of an article, an assembled article, and a schematic showing how power is consumed by the device, respectively, according to some embodiments;

FIG. 21A shows the current consumption at various power levels for articles, according to some embodiments;

FIG. 21B shows signal strength of an antenna in water as a function of case medium, according to some embodiments;

FIG. 22 shows received wireless test signals from an article, according to some embodiments;

FIGS. 23A-23C show the placement of an article in-vivo, according to some embodiments;

FIGS. 24A-24B show raw measurements from an in-vivo study using an article, according to some embodiments;

FIGS. 25A-25C show multimodal measurements received from an article, according to some embodiments;

FIG. 26 is a schematic illustration showing how an article may be placed in-vivo, according to some embodiments;

FIG. 27 is a schematic illustration showing how an article may be used as a function of time, according to some embodiments; and

FIG. 28 shows data collected using an article over multiple days, according to some embodiments.

### DETAILED DESCRIPTION

Some aspects of the present disclosure are related to articles for measuring electrical signals internal to a subject, e.g., of the gastrointestinal tract (GI tract). The articles, in some embodiments, are ingestible and/or implantable. According to some embodiments, the articles comprise a substrate comprising a plurality of electrodes, the substrate having a Young's elastic modulus of greater than or equal to 0.01 MPa and less than or equal to 200 MPa. In some such cases, the Young's elastic modulus of the substrate is selected to facilitate elastic recoil of the substrate such that at least a portion of the plurality of electrodes contact a surface of a tissue at the location internal to the subject (e.g., the GI tract) and thus facilitate measurement of electrical signals at the location internal to the subject. Other aspects are related to methods of using the articles, for example, to monitor electrical signals from the location internal to the subject.

Measuring electrical signals internal to the body can provide information about the functioning of various organs and/or systems within the body. For example, electrical activity associated with the GI tract (e.g., from neurons associated therewith) regulates motility and hormone secretion, and dysfunction or other irregularities with such signals may indicate various GI disorders, such as gastroparesis. However, measuring such electronic signals within the body is challenging due to the depth within the body from which the signals originate. Advantageously, some aspects of the present disclosure are related to articles that measure electrical signals that propagate internal to a subject for monitoring electrical activity within a subject (e.g., of the GI tract).

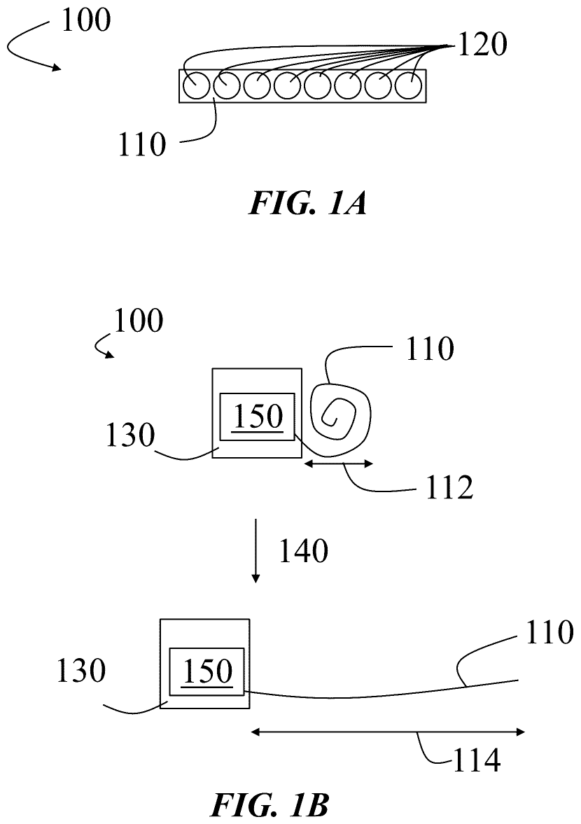

Some aspects are related to articles for monitoring electrical signals at a location internal to a subject. In some embodiments, the article comprises a substrate comprising a plurality of electrodes, the article sized and adapted for ingestion and/or implantation at the location internal to the subject. In some such embodiments, the substrate has a Young's elastic modulus of greater than or equal to 0.01 MPa and less than or equal to 200 MPa, wherein at the location internal to the subject, the substrate undergoes elastic recoil sufficient such that at least a portion of the plurality of electrodes contact a surface of a tissue at the location internal to the subject. For example, FIG. 1A is a schematic diagram showing an article 100 comprising substrate 110 comprising a plurality of electrodes 120.

The article comprises a substrate. The substrate, in accordance with some embodiments, comprises an elastic polymeric material. In some embodiments, the substrate comprises polyimide. In some embodiments, the substrate comprises a flexible printed circuit board.

The elastic polymeric material is preferably biocompatible. The term “biocompatible,” as used in reference to a polymeric component and/or other materials of the article, refers to a polymer or other material that does not invoke a substantial adverse reaction (e.g., deleterious immune response) from an organism or subject (e.g., a mammal), a tissue culture or a collection of cells, or invokes only a reaction that does not exceed an acceptable level.

In some embodiments, the substrate is shaped and configured such that it may assume a first configuration and a second configuration as described elsewhere herein. In some embodiments, the substrate is shaped such that the plurality of electrodes adjacent to the substrate may be spaced and configured to acquire an electrical signal between the electrodes over a certain distances. In some embodiments, the substrate is shaped and sized to accommodate the plurality of electrodes. For example, the plurality of electrodes may be spaced on the substrate such that the plurality of electrodes spans a distance of greater than or equal to 1 cm, greater than or equal to 2 cm, greater than or equal to 3 cm, greater than or equal to 5 cm, greater than or equal to 8 cm, greater than or equal to 10 cm, greater than or equal to 15 cm, greater than or equal to 20 cm, greater than or equal to 25 cm, greater than or equal to 30 cm, or greater than or equal to 40 cm. In some embodiments, the plurality of electrodes may be spaced on the substrate such that the plurality of electrodes spans a distance of less than or equal to 50 cm, less than or equal to 40 cm, less than or equal to 30 cm, less than or equal to 20 cm, less than or equal to 10 cm, less than or equal to 8 cm, less than or equal to 5 cm, less than or equal to 3 cm, or less than or equal to 2 cm. Combinations of the foregoing ranges are possible (e.g., greater than or equal to 1 cm and less than or equal to 50 cm). Other ranges are also possible.

The substrate may have any of a variety of dimensions, according to some embodiments. In some embodiments, the dimensions of the substrate may be selected to facilitate the substrate being similarly sized or smaller to a capsule, e.g., in a first configuration, as described elsewhere herein. In some embodiments, the substrate is sized and adapted for ingestion and/or implantation at the location internal to the subject. In some embodiments, the length and/or width of the substrate greater than or equal to 1 mm, greater than or equal to 1 cm, greater than or equal to 10 cm, greater than or equal to 20 cm, greater than or equal to 30 cm, or greater than or equal to 40 cm. According to some embodiments, the length and/or width of the substrate may be less than or equal to 50 cm, less than or equal to 40 cm, less than or equal to 30 cm, less than or equal to 20 cm, less than or equal to 10 cm, or less than or equal to 1 cm. Combinations of the foregoing ranges are possible. Other ranges are also possible. In some embodiments, a thickness of the substrate is less than or equal to 3 mm, less than or equal to 2 mm, less than or equal to 1 mm, less than or equal to 500 microns, less than or equal to 100 microns, less than or equal to 50 microns, or less than or equal to 20 microns.

As described elsewhere herein, in some embodiments, an aspect ratio of the substrate is selected to facilitate the placement of the plurality of electrodes such that the plurality of electrodes may contact a tissue at a location internal to the subject over a variety of distances. In some embodiments, the substrate has an aspect ratio (e.g., a length:width aspect ratio) of greater than or equal to 2, greater than or equal to 5, greater than or equal to 10, greater than or equal to 20, greater than or equal to 30, greater than or equal to 40, greater than or equal to 50, or greater than or equal to 75. In some embodiments, the aspect ratio of the substrate is less than or equal to 100, less than or equal to 75, less than or equal to 50, less than or equal to 40, less than or equal to 30, less than or equal to 20, less than or equal to 10, or less than or equal to 5. Combinations of the foregoing ranges are possible (e.g., greater than or equal to 2 and less than or equal to 100). Other ranges are also possible.

The article comprises a plurality of electrodes, in accordance with some embodiments. In some embodiments, the substrate comprises the plurality of electrodes. In accordance with some embodiments, at least a portion of the plurality of electrodes are adjacent the substrate. As used herein, when a component is referred to as being “adjacent” another component, it can be directly adjacent to (e.g., in contact with) the component, or one or more intervening components (e.g., a liquid, a hollow portion) also may be present. A component that is “directly adjacent” another component means that no intervening component(s) is present. In accordance with some embodiments, the plurality of electrodes is adjacent the substrate. In accordance with some embodiments, at least a portion of the plurality of electrodes are formed over the substrate, e.g., with one or more intervening layer between the substrate and the at least a portion of the plurality of electrodes. In some embodiments, at least a portion of the plurality of electrodes are formed directly on the substrate. In some cases, all of the plurality of electrodes are formed over and/or formed on the substrate. In some cases, all of the plurality of electrodes are formed directly on the substrate.

The electrodes may comprise any of a variety of materials, as long as the material is suitable for use within a subject and the material is conductive. Suitable for use within a subject generally indicates that the material is non-toxic and/or biocompatible. For example, the material may be present at a location internal to a subject without negatively altering the health or condition of the subject, relative to the health or condition of the subject without the material being present. Example materials include inert conductors, e.g., gold, platinum, carbon, titanium, and some conductive polymers (e.g., polypyrrole and/or polythiophene).

The plurality of electrodes may contain any of a variety of number, geometries, and/or sizes of electrodes, in accordance with some embodiments. In some embodiments, the plurality of electrodes comprises at least 2, at least 3, at least 4, at least 5, at least, 6, at least, 7, at least 8, at least 9, at least 10 electrodes, and so forth. For example, again referring to FIG. 1A, the substrate 110 comprises a plurality of electrodes 120 comprising 8 electrodes. The number of electrodes within the plurality may be adjusted based on the region internal to the subject where the electrical signals are to be measured and/or the dimensions of the substrate, in some embodiments. In some embodiments, the number of electrodes may be adjusted based on the other electronic components, e.g., that can be housed within the capsule.

The electrodes may be sized and spaced to minimize the size of the substrate and/or article, e.g., to facilitate the substrate assuming a first configuration and/or internalization of the article, in some embodiments. In some embodiments, the electrodes may have a largest average dimension of greater than or equal to 1 mm, greater than or equal to 5 mm, or greater than or equal to 1 cm. In some embodiments, the electrodes may have a largest average dimension or less than or equal to 2 cm, less than or equal to 1 cm, or less than or equal to 5 mm. combinations of the foregoing ranges are possible. Other ranges are also possible. In some embodiments, the average spacing between two adjacent electrodes may be greater than or equal to 1 mm, greater than or equal to 5 mm, greater than or equal to 1 cm, greater than or equal to 2 cm, or greater than or equal to 3 cm. In some embodiments, the average spacing between two adjacent electrodes between two adjacent electrodes may be less than or equal to 5 cm, less than or equal to 3 cm, less than or equal to 2 cm, less than or equal to 1 cm, or less than or equal to 5 mm. Combinations of the foregoing ranges are possible (e.g., greater than or equal to 1 mm and less than or equal to 5 cm). Other ranges are also possible. In some embodiments, the electrodes may be in electrical communication with other electrodes and/or electronic components of the articles described herein, e.g., by wires present on the substrate as described elsewhere herein. In some such embodiments, the dimensions of the electrodes as described above do not consider the wires established electrical communication. In some embodiments, the thickness of the electrodes is less than or equal to 1 mm, less than or equal to 500 microns, less than or equal to 100 microns, less than or equal to 50 microns, or less than or equal to 20 microns. It will be understood by those of ordinary skill in the art, based upon the teachings of this specification, that the thickness of the electrodes may be selected to maintain a certain conductivity therethrough.

In some embodiments, the article further comprises electronic components, for example, a microcontroller, a transceiver, an antenna, and/or a processor. In some embodiments, the article further comprises a power source, e.g., a battery. Other electronic components are also possible. Some and/or all of the electronic components may be present on and/or in electrical communication with a circuit board, for example, a printed circuit board. In some such embodiments, the printed circuit board may be in electrical communication with the substrate and/or the plurality of electrodes. In some embodiments, the plurality of electrodes is in electrical communication with one or more electronic components, e.g., a processor, a transceiver, etc. Electrical communication between the plurality of electrodes and the electronic components may be advantageous, for example, to facilitate the use of the plurality of electrodes to determine an electrical signal at a location internal to the stomach and/or to transmit data corresponding to the determined electrical signal, as described elsewhere herein. In some such embodiments, the electrical signal determined with the plurality of electrodes is correlated with a gastric slow wave and/or gastric motility. In some embodiments, each of the plurality of electrodes may be electrically connected to other electronic components present in the article. In some such embodiments, electrical connections may be facilitated by wires, e.g., soldered wires or lithographically fabricated wires between the electrodes and/or other electronic components. Other methods suitable for electrically connecting the electrodes to other electronic components are also possible.

In some embodiments, the article may further comprise a capsule or other suitable housing or vessel, for example, for containing other electronic components. In some embodiments, some or all of the other components of the article may be contained within the capsule or other suitable housing or vessel. For example, in some embodiments, the substrate is disposed within the capsule. As described below, the substrate may be in a first configuration when disposed within the capsule. In some embodiments, the substrate is not disposed within the capsule. For example, in accordance with some embodiments, the substrate is only partially disposed within the capsule. For instance, in some embodiments, only a first portion of the substrate is disposed within the capsule, where a second portion remains outside the capsule.

The capsule or other suitable vessel may be made of any of a variety of biocompatible materials, in accordance with some embodiments. In some embodiments, the capsule may comprise a polymer, e.g., a rigid polymer. Other materials for the capsule are also possible.

In some embodiments, the capsule may be sized and adapted for ingestion. In some embodiments, the capsule may be sized such that it is in line with FDA-approved products for endoscopy systems and/or pills. For example, in some embodiments, the largest dimension of the capsule (e.g., a length, a width, or a thickness) is no more than 25 mm, no more than 22 mm, no more than 20 mm, no more than 18 mm, no more than 15 mm, no more than 12 mm, or no more than 10 mm. In some embodiments, the dimensions of the capsule correspond to or are smaller than a 000-sized pill. In some embodiments, the dimensions of the capsule correspond to or are smaller than a 00-sized pill. In some embodiments, sizes of the capsule corresponding to smaller pill sizes may be desirable to facilitate ingestion, e.g., by swallowing. Larger pill sizes are also possible, e.g., corresponding to larger veterinary capsules such as Su07, 7, 10, 12e1, 11, 12, 13, 110 ml, 90 ml, and 36 ml. It will be appreciated that the selected size of the capsule may be determined based on the identity of the subject. Larger sizes (e.g., corresponding to 000- or 00-pill sizes or larger) may facilitate the inclusion of additional and/or larger components to the article, e.g., a larger plurality of electrodes, larger and/or additional electronic components, a larger battery, etc. Including additional electronic components, in some embodiments, may facilitate additional functionalities of the article. For instance, additional electronic components may be included to improve communication between the article and a receiver.

The material of the substrate may have any of a variety of suitable Young's elastic moduli, according to some embodiments. In some cases, the substrate has an elasticity that facilitates elastic recoil, for example, to change a configuration of the substrate. In accordance with some embodiments, as described in more detail elsewhere herein, the substrate may be configured to change from a first, closed configuration to a second, open configuration. In some embodiments, the elastic recoil associated with the Young's elastic modulus of the substrate facilitates the change from the closed configuration to the open configuration. Advantageously, in some such embodiments, the elasticity and related changing of the configuration facilitates the contacting of the plurality of electrodes of the substrate with a tissue at the location internal of the subject and thus the monitoring of electrical signals that propagate throughout the tissue. It will be understood that, in some embodiments, the elasticity and related changing of the configuration facilitates the contacting of at least a portion of the plurality of electrodes of the substrate with a tissue at the location internal of the subject, for example, at least two electrodes of the plurality of electrodes. Moreover, in some embodiments, the Young's elastic modulus is selected such that the substrate maintains some flexibility to conform to the shape of the location internal to the subject (e.g., the lining of the stomach) and to contact tissues therein. For example, a Young's modulus of the substrate being above 200 MPa would be detrimental to the articles described herein, as the substrate would not conform to the location internal to the subject and would not be able to contact the tissue with at least a portion of the plurality of electrodes. Furthermore, having a Young's modulus of the substrate above 200 MPa may lead to mechanical damage by the article to the internal location of the subject and/or may prevent the article clearing from the location internal to the subject (e.g., via gastric clearance).

In some embodiments, the Young's elastic modulus of the substrate is greater than or equal to 0.01 MPa greater than or equal to 0.1 MPa, greater than or equal to 0.2 MPa, greater than or equal to 0.3 MPa, greater than or equal to 0.5 MPa, greater than or equal to 1 MPa, greater than or equal to 2 MPa, greater than or equal to 5 MPa, greater than or equal to 10 MPa, greater than or equal to 25 MPa, greater than or equal to 50 MPa, or greater than or equal to 100 MPa. In some embodiments, the Young's elastic modulus of the substrate is less than or equal to 200 MPa, less than or equal to 100 MPa, less than or equal to 50 MPa, less than or equal to 25 MPa, less than or equal to 10 MPa, less than or equal to 5 MPa, less than or equal to 2 MPa, less than or equal to 1 MPa, less than or equal to 0.5 MPa, less than or equal to 0.3 MPa, less than or equal to 0.2 MPa, or less than or equal to 0.1 MPa. Combinations of the foregoing ranges are possible (e.g., between 0.01 MPa and 200 MPa, between 0.3 MPa and 10 MPa). Other ranges are also possible. Those skilled in the art would be capable of selecting suitable methods for determining the elastic modulus of the substrate including, for example, compressive mechanical characterization under ASTM D575.

In some embodiments, the substrate and the plurality of electrodes may comprise a first configuration, for example, when before being internalized. In accordance with some embodiments, the first configuration is closed. In some embodiments, when the substrate is in its first configuration, it is folded, rolled, bent, and/or otherwise compressed. In some embodiments, the substrate may be in a first configuration to fit within the capsule. In some embodiments, the substrate may be in a first configuration to facilitate internalization thereof into a subject. FIG. 1B is a schematic showing a side profile view of an article 100 including a substrate 110 in a first configuration such that is adjacent to capsule 130. In this instance, the thickness of the electrodes of the plurality of electrodes is relatively thin compared to the thickness of the substrate, and thus the plurality of electrodes is not pictured. The substrate is in electrical communication with electronics 150 housed in capsule 130. It will be understood that the embodiment depicted in FIG. 1B is nonlimiting and that the relative thicknesses of the substrate and electrodes may vary. For instance, the thickness of the electrode may be comparable and/or relatively thick when compared to the substrate, in some embodiments.

In some embodiments, the substrate, upon contact with a fluid at the location internal to the subject and/or release from a capsule following such contact with the fluid, may change from a first configuration to a second configuration. In some embodiments, the second configuration is different from the first configuration. In some embodiments, the second configuration is open. In some embodiments, when the substrate is in its second configuration, it is unrolled, unbent, unfolded, or otherwise expanded such that its effective length is longer than the effective length in its first configuration. Effective length is the length observed when in a certain configuration, and not necessarily the length of the substrate (e.g., or other component) when fully extended. It will be understood that the effective length may correspond wot the length of the substrate when fully extended, in some embodiments. FIG. 1B shows a side view of the article undergoing elastic recoil and changing from a first configuration to a second configuration 140 when the substrate is internalized, e.g., and contacts a fluid at a location internal to the subject. FIG. 1B further illustrates that the substrate has an effective length of 112 when in its first configuration and expands to have an effective length of 114 in its second configuration. Additionally, FIG. 1B shows that the capsule may further contain other components, e.g., electronic components 150. As depicted, the electronic components 150 may remain within the capsule following internalization of the article 100. Accordingly, in some such embodiments, the substrate comprising the plurality of electrodes may remain connected (e.g., mechanically and/or electronically) to the capsule and/or the other components, as shown in FIG. 1B, even when in its second configuration is attained by the substrate.

FIG. 1B additionally depicts that at least a portion of the substrate 110 may extend through at least a portion of the capsule 130 such that the substrate 110 may be mechanically and/or electronically connected to other electronic components 150 contained within the capsule 130. In some instances, the capsule may be configured for the substrate to the extend therethrough while also maintaining preventing exposure of electronic components contained therein to conditions outside the capsule (e.g., physiological conditions when the capsule is internalized by a subject).

In some embodiments, as described above, the Young's elastic modulus of the substrate may facilitate elastic recoil such that the substrate changes from a first configuration to a second configuration, different from the first configuration, when released from the capsule and/or upon contact with a fluid at the location internal to the subject. A nonlimiting example of the substrate changing from a first configuration to a second configuration as a result of elastic recoil is depicted in FIG. 1B, which is described in more detail elsewhere herein. In some embodiments, at least a portion of the plurality of electrodes of the substrate contact a surface of a tissue at the location internal to the subject when the substrate assumes its second configuration.

Some aspects are related to methods. For example, in some embodiments, the method is related to monitoring electrical signals at a location internal to a subject using the articles as described elsewhere herein. In some embodiments, the method comprises contacting a surface of a tissue with at least a portion of a plurality of electrodes of the article at the location internal to the subject. In some embodiments, the method further comprises using the at least a portion of the plurality of electrodes to determine an electrical signal, wherein the electrical signal is correlated with a gastric slow wave and/or gastric motility. For instance, in some embodiments, the method includes contacting a surface of a tissue with at least two electrodes of the plurality of electrodes of the article at the location internal to the subject.

In some embodiments, as described elsewhere herein, the article may be used to contact a tissue at a location internal to a subject. The term “subject,” as used herein, refers to an individual organism such as a human or an animal. In some embodiments, the subject is a mammal (e.g., a human, a non-human primate, or a non-human mammal), a vertebrate, a laboratory animal, a domesticated animal, an agricultural animal, or a companion animal. In some embodiments, the subject is a human. In some embodiments, the subject is a rodent, a mouse, a rat, a hamster, a rabbit, a dog, a cat, a cow, a goat, a sheep, or a pig. Additionally, those of ordinary skill in the art will understand the meaning of a location internal to the subject. It will be further understood that the size and configuration of the article may be altered based on the identity of the subject. For instance, a larger article may be used when the subject is a human when compared to an article configured for used in a mouse.

In some embodiments, the articles described herein are suitable for measuring electrical signals at various locations internal to a subject, as long as at least a portion of the plurality of electrodes contact at least a portion of a tissue at the location. In some embodiments, the location internal to the subject is the stomach of the subject.

In some embodiments, the method further comprises contacting tissue at the location internal to the subject. In some such embodiments, contacting tissue at the location internal to the subject comprises at least a portion of the plurality of electrodes contacting the tissue at the location internal to the subject. In some embodiments, contacting the tissue with at least a portion of the plurality of electrode completes a circuit comprising the plurality of electrodes, thereby facilitating the measurement of electrical signals propagating through the tissue that is contacted with the plurality of electrodes.

In some embodiments, the tissue that is contacted is gastric tissue. In some embodiments, the tissue that is contacted internal to the subject comprises other tissues of the gastrointestinal tract. In this manner, in some embodiments, as the at least a portion of the plurality of electrodes contacts the tissue, electrical signals passing through and/or nearby to the gastrointestinal tract (e.g., through the stomach, through other organs adjacent to the GI tract such as the heart, lungs, etc.) may be determined using the at least a portion of the plurality of electrodes contacting the tissue. In some embodiments, the at least a portion of the plurality of electrodes contacting the tissue used to determine an electrical signal passing through and/or nearby to the GI tract. In some embodiments, the method comprises using the at least a portion of the plurality of electrodes to determine an electrical signal that is correlated with a gastric slow wave and/or gastric motility.

The method may further include acquiring data from the article. In accordance with some embodiments. In some embodiments, due to the article being present at the location internal to the subject, acquiring data from the article is done remotely. For example, in some embodiments, the article wirelessly transmits data from the location internal to the subject. In some embodiments, the data is associated with the electrical signal measured by the at least a portion of the plurality of electrodes of the article contacting the tissue. In some such embodiments, the electrical signal is correlated with a gastric slow wave and/or gastric motility. Accordingly, in some embodiments, the article determines an electrical signal, e.g., using at least a portion of the plurality electrodes contacting a tissue at a location internal to a subject, whereafter the electrical signal is wirelessly transmitted as data to a location external to the subject. In some embodiments, the data is received external the subject. In some embodiments, a receiver is present external to the subject to receive the data transmitted by the article. In some embodiments, acquiring data may comprise wirelessly transmitting data from the article to a receiver external to the subject.

In some embodiments, in order to facilitate the measurement and/or determination of the electrical signal, and/or the transmission of the data that is associated with the electrical signal, the article may further comprise any of a variety of electrical components. For example, the article may further comprise microelectronics and/or a wireless power source such as a battery. In some embodiments, with the microelectronics may be present on a printed circuit board, and comprise an analog to digital controller (e.g., an 8-channel ADC), a microcontroller, a transceiver, and/or an antenna. Other electronic components are possible and may be selected by those of ordinary skill in the art to facilitate different measurements and/or communication routes by the article. In some embodiments, the battery acts as a wireless power source, and thus facilitates the use of the article at the location internal to the subject.

In some embodiments, the methods comprise contacting a surface of a tissue with at least a portion of the plurality of electrodes of the article at the location internal to the subject. In some embodiments, the method may comprise internalizing the article comprising the plurality of electrodes. In some embodiments, internalizing the article may occur before contacting a surface of a tissue with at least a portion of the plurality of electrodes of the article at the location internal to the subject. Internalizing the article, in some embodiments, may occur in various methods. In some embodiments, the article may be implanted within a subject, e.g., by using an endoscope. Implanting may be facilitated by endo-clips which are configured to maintain a position of at least a portion of the article, in some embodiments. Articles configured to be implanted, according to some embodiments, may not comprise a capsule. It will be understood, in other embodiments, the article to be implanted may still include a capsule or other suitable housing or vessel, e.g., for containing one or more electronic components thereof. In some embodiments, implanting may be desirable as it may facilitate longer term retention of the article than when the article is internalized via other methods.

According to some embodiments, internalizing the article may comprise ingesting the article by a subject, e.g., by swallowing the article. In some such embodiments, the article may comprise a capsule. Advantageously, in some such embodiments wherein the method comprises ingesting the article, the method may facilitate the internalization of the article in a non-invasive way. Accordingly, in some embodiments wherein the article is configured to determine an electrical signal that is associated with a gastric slow wave and/or gastric motility, ingesting the article provides a quantitative and non-invasive method to facilitate such determining.

In some embodiments, upon internalization, the article changes configurations from the first configuration to a second configuration. In some embodiments, upon implantation, the article changes configurations from the first configuration to a second configuration. In some embodiments, upon ingestion, the article changes configurations from the first configuration to a second configuration. In some embodiments, upon with a fluid at the location internal to the subject, the article changes configurations from the first configuration to a second configuration. In some embodiments, the first configuration is closed, for example, some and/or all of the components of the article are contained within a capsule, as described elsewhere herein. Following internalization of an article comprising a capsule, according to some embodiments, the substrate may change from its first configuration to assume a second configuration. In some embodiments, the second configuration is open, for example, at least some of the components of the article are located external of the capsule. Accordingly, in some embodiments, the article may change configurations from the first configuration to the second configuration at a location in internal to the subject. In some embodiments, the method comprises internalizing the article comprising a substrate having a first configuration at the location internal to the subject such that the substrate recoils to obtain a second configuration that is different from the first configuration. In some embodiments, the method includes allowing the substrate of the article to change from a first configuration to a second configuration. In some such embodiments, the changing of the configuration of the substrate of the article occurs following internalizing the article.

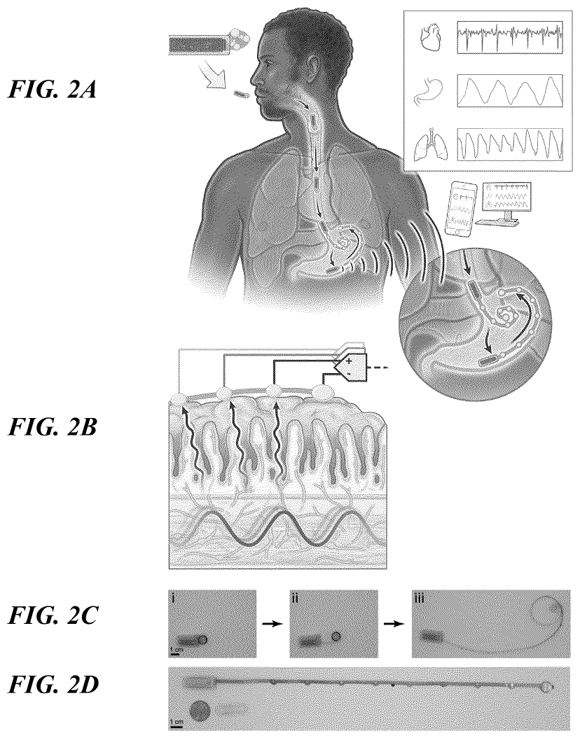

In some embodiments, the substrate of the article may be in the first configuration when internalized to facilitate the internalization of the article (e.g., swallowing of the article, implantation of the article). In some embodiments, the substrate of the article may be in the first configuration when ingested to facilitate the ingestion of the article (e.g., swallowing of the article). In some embodiments, following ingesting, the substrate of the article may change from its first configuration to its second configuration. Again, referring to FIG. 1B, the schematic diagram shows an example of a substrate within a capsule in a first configuration changing to a second configuration. In some embodiments, when the substrate comprises its second configuration at the location internal of the subject, the plurality of electrodes of the substrate of the article may be exposed to the subject. In some embodiments, at least a portion of the plurality of electrodes may contact a tissue at the location internal of the subject, as described above and shown in FIG. 2A.

In some embodiments, the at least a portion of the plurality of electrodes comprises at least two electrodes of the plurality of electrodes, thereby completing a circuit between the at least two electrodes. In some embodiments, more than two electrodes, such as three electrodes, four electrodes, five electrodes, and so forth, up to all of the electrodes of the plurality of electrodes of the substrate may contact the tissue, whereafter the article may determine an electrical signal at the location internal of the subject.

In some embodiments, the article has a size adapted and configured such that, after contacting a surface at a location internal to a subject, the article exits the location internal to the subject. In some embodiments, prior to exiting the location internal to the subject, the article may be retained at the location internal to the subject, for example, at least 1 minute, at least 5 minutes, at least 15 minutes, at least 30 minutes, at least 1 hour, or at least 6 hours, and/or up to 12 hours, or up to 1 day. In some such embodiments, retention may be facilitated, e.g., by a endo-clips configured to maintain a location of the article at the location internal to the subject. It will be understood that endo-clips may be present, e.g., when the article is implanted in the subject, in some embodiments. In an exemplary set of embodiments, and without wishing to be limited by such, in some embodiments, after contacting a tissue at a location internal to the subject such as the stomach, the article passes through the pylorus thereby exiting the location internal to the subject. In some embodiments, the article (e.g., in the second configuration) is sized and adapted to safely transit the small and/or large intestine of the subject.

The following examples are intended to illustrate certain embodiments of the present invention, but do not exemplify the full scope of the invention.

### Example 1

The following example describes a system for multimodal electrophysiology via ingestible, gastric, untethered tracking, as well as experiment testing the function of the system.

Despite growing interest in the gut-brain axis, gastrointestinal electrophysiology in large mammals remains understudied due to the invasiveness of placing high-fidelity systems without limiting subject movements. Electrodes may, in some cases, either be implanted surgically or worn on the skin resulting in a tradeoff between signal quality and invasiveness. In this example, a system for multimodal electrophysiology via ingestible, gastric, untethered tracking (MiGUT) is introduced, which can record electrical signals in the stomach. MiGUT can record distinct signals including the slow wave and other gastric electrical activity in the stomach, as well as the heart and respiration signals due to its proximity to associated vital organs. Moreover, MiGUT can monitor slow wave activity in freely moving and feeding animals, demonstrating a new concept for ingestible electronics and the study of gastric electrophysiology.

### INTRODUCTION

The enteric nervous system (ENS) contains millions of neurons and associated electrically active cells which regulate motility and hormone secretion in the gastrointestinal (GI) tract. Dysfunction in electrical signaling is associated with a wide range of debilitating GI disorders; examples include gastroparesis, which can arise either idiopathically or as a complication of diabetes, and functional dyspepsia which has an estimated worldwide prevalence of 10-30%. There is also mounting evidence of a gut-brain axis dysfunction in many neurological disorders; patients who develop Parkinson's are reported to exhibit early GI motility issues and gastrointestinal issues are widespread in those with autism spectrum disorder. Monitoring of baseline GI electrophysiology is limited, which makes it difficult to differentiate changes due to regular daily activities from those arising from disease pathology. Consequently, methods which can interrogate the electrical signaling of the ENS throughout the GI tract with high fidelity can both advance fundamental understanding of these disorders and improve healthcare via differential and/or early disease diagnosis.

Effectively capturing GI biopotentials has remained a challenge due to the depth of the signal's origin within the GI tract. In the context of the stomach, with its 3-4 cycles/min gastric slow wave, cutaneous electrogastrography (EGG) was discovered in the early 20th century but was not widely adopted due to attenuation of the signal through the abdominal tissue and artifacts arising from motion or myoelectric activity. High resolution mapping via serosal surgical placement of multi-electrode arrays using laparotomy are able to obtain high quality recordings, but are significantly more invasive and typically only conducted under anesthesia which can change gastric electrical activity. Gastric mucosal recordings have been shown to exhibit comparable quality to serosal recordings, facilitating less invasive measurements, but electrodes are usually tethered endoscopically for acute measurements or require nasally clipped leads which are passed through the esophagus to an external reader unit for awake recordings.

Recently, significant advances in EGG signal processing using artifact rejection algorithms have been made but there exists a key unmet need for devices which can record high quality electrophysiology data in the GI tract and ENS without any limitations to subject motion and/or comfort.

Untethered, ingestible electronic sensors have been of increasing interest as a non-invasive method to monitor physiological signals as they can be orally delivered and measure relevant information such as core temperature, pressure, GI metabolites and gas concentration. Moreover, ingestible devices cause minimal disruption to the biology of the GI tract as their placement does not require surgery or cause any damage to GI tissue. Motivated by these advantages, the challenges of studying GI electrophysiology by developing a device for Multimodal electrophysiology via ingestible, Gastric, Untethered Tracking (MiGUT) is addressed. MiGUT comprises encapsulated electronics 200 and a battery 210 (FIG. 2A), as well as a sensing electrode ribbon stored in a rolled configuration (FIGS. 2C and 2D). Following delivery, the electrodes unroll in the stomach to make contact with the mucosa (FIG. 2B) and the device records biopotential signals which are subsequently transmitted wirelessly to an external receiver several meters away. The location of the MiGUT device when placed in the stomach facilitates determination of electrical activity in the vital organs within close proximity. In this example, it is demonstrated that MiGUT can record high quality biopotential signals, including the gastric slow wave, respiration rate, electrocardiogram, as well as putative signals related to the migrating myoelectric complex in a large animal model. Moreover, it is shown that the device can be transiently fixed to the gastric mucosa using endoscopic clips to measure and wirelessly transmit signals from the gastric slow wave during feeding, sleeping and ambulation over multiple days.

### Device Design and Characterization

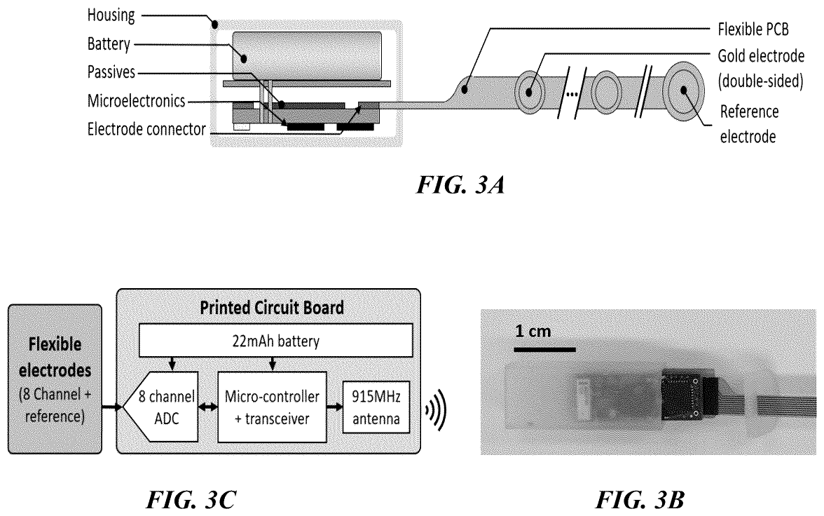

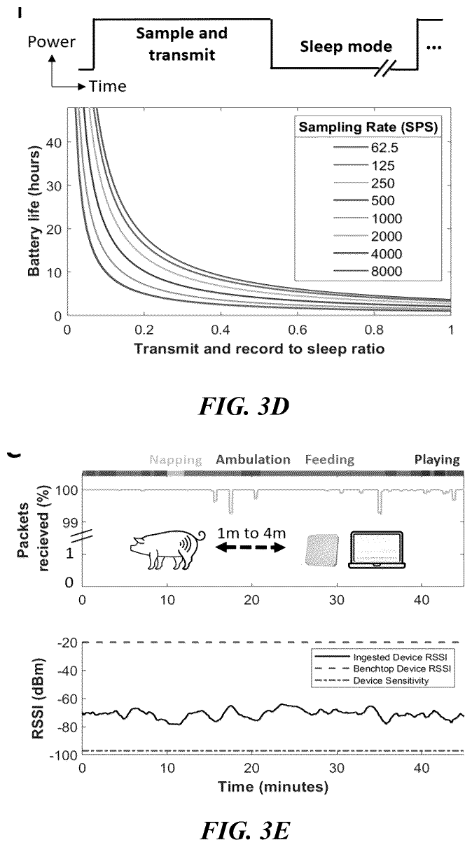

The MiGUT system shown in FIG. 3A consists of an electronics module and flexible measurement electrodes. The electronics are housed in a 9×12×26 mm³ 3D printed case (FIG. 3B) which was designed to be comparable to FDA cleared products such as capsule endoscopy systems to facilitate future clinical translation. Biopotentials are measured using an eight channel 16-bit analog-to-digital converter (ADC) and are wirelessly transmitted to a base station using a microcontroller with integrated 915 MHz transceiver (FIG. 3C). The current consumption of the device depends on the sampling frequency, providing flexibility in balancing experiment length and signal fidelity. Duty cycling (Methods) between measurement mode and low power sleep mode facilitates experiments to be conducted over a number of days (FIG. 3D). With a panel antenna approximately one to four meters away (Methods) from the device in a freely moving swine (97 kg), an external 915 MHz transceiver was found to reliably receive over 99% of data from the MiGUT system at approximately −70 dBm in a full stomach and during multiple behaviors (FIG. 3E).

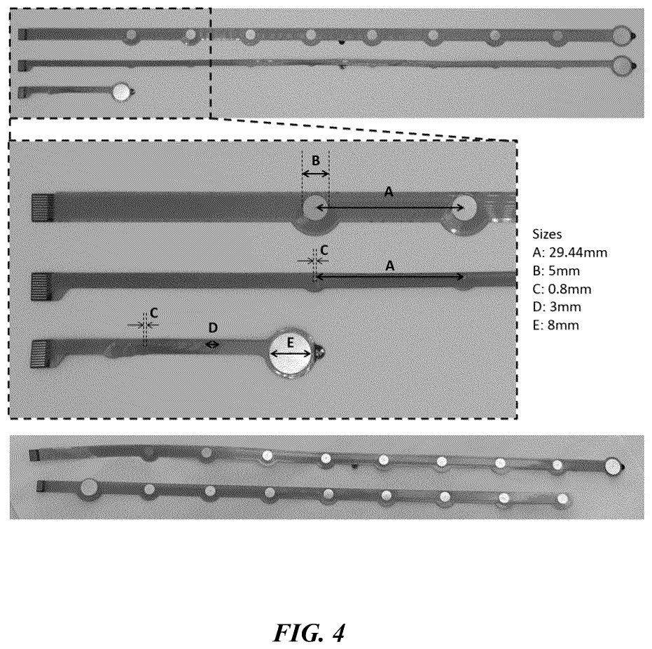

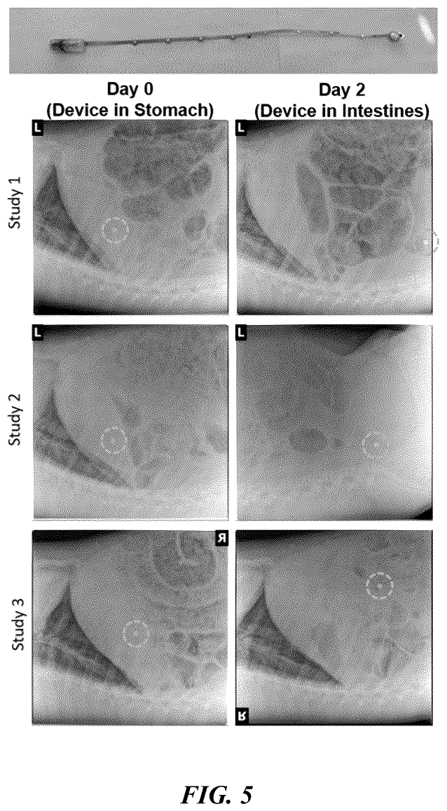

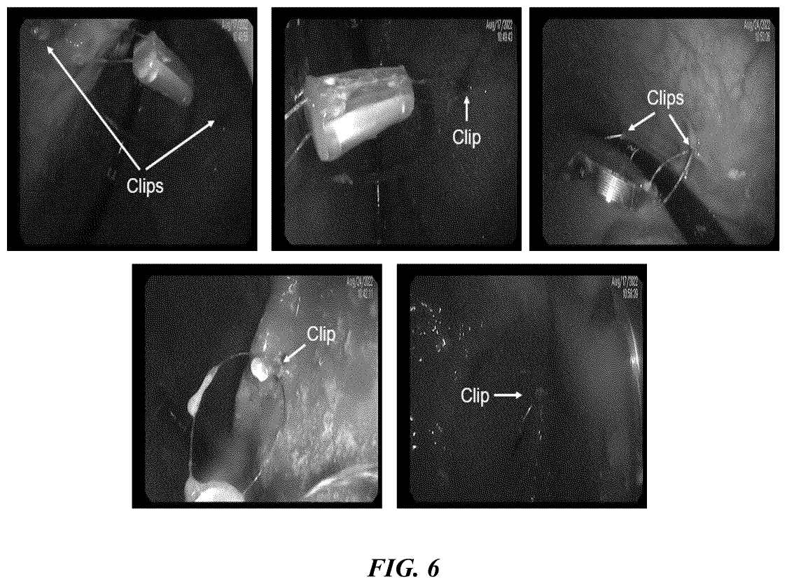

A flat flexible cable connector on the electronics module facilitates a range of flexible electrode designs that can be used with the system, such as custom designed electrodes fabricated using flexible printed circuit board (PCB) manufacturing methods (FIG. 4, Methods). In particular, a polyimide ribbon (total thickness 75 um) of 25 cm in length containing eight gold recording electrodes and one reference electrode (double sided, ø5 mm and ø8 mm respectively) was developed specifically to span the greater curvature of the stomach and conform to the mucosa facilitating gastric recordings. The electrode was assembled in a rolled configuration secured with water soluble adhesive around a circular roller (Methods, FIGS. 2C and 2D). Once MiGUT comes into contact with gastric fluid following oral delivery to the stomach, the electrode unrolls due to inbuilt strain in the rolled ribbon along with the mechanical motion of the stomach. Safety of the MiGUT system was confirmed in passage studies showing that following delivery to the porcine stomach, the combined electronics and electrode ribbon system passes out of the animal by day 5 (N=3, FIG. 5). For longer experiments >24 hrs, the device can be clipped to the mucosa (FIG. 6, N=5) in a manner similar to that reported in literature supporting evaluation during solid food ingestion.

To demonstrate the capabilities of the MiGUT system in an in-vivo porcine model, a set of experiments was designed to (i) record and assess signal components in anesthetized animals, (ii) assess inducted gastric electrophysiology changes with introduction of a gastric motility modulating compound in anesthetized animals, and (iii) demonstrate recording in fully awake and ambulating animals.

### Multimodal Measurements in Anesthetized Animals

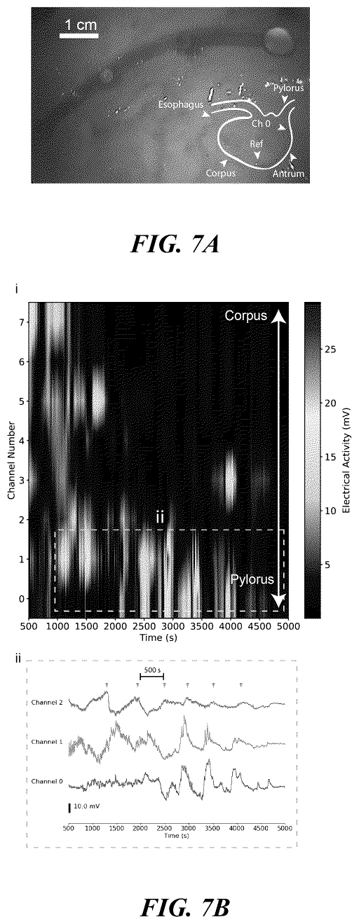

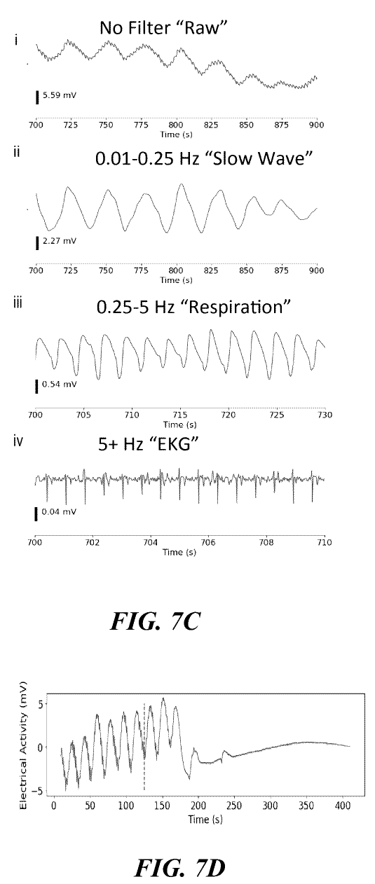

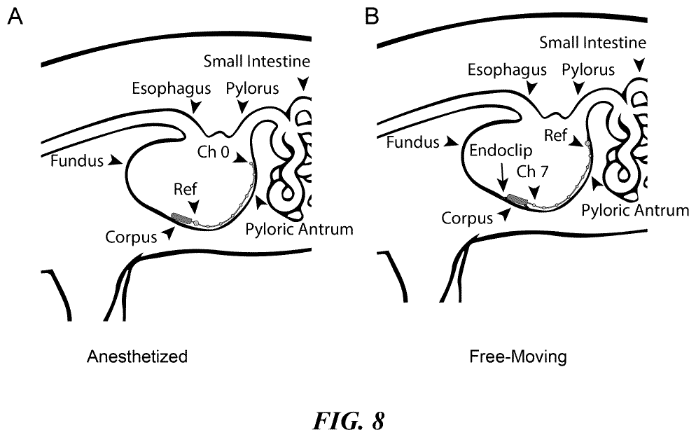

In an anesthetized Yorkshire pig, the device was delivered into the stomach and the electrode is shown to unroll and conform to the gastric mucosa along the greater curvature of the stomach (FIG. 7A). The reference electrode was localized near the stomach corpus (Schematic in FIG. 8). One and a half hours of recorded data in this experiment (at 62.5 samples/s) was used to generate a heat map of electrical activity vs time and channel (FIG. 7B(i)). Initial electrical activity seen in all channels following device placement (t=0 to 500 seconds) is most likely due to mechanical stimulation as a consequence of endoscopic visualization following device deployment. Channels near the pylorus (FIG. 7B(ii), channels 0-2) measured large amplitude, prolonged waveforms with periods of ca. 500 seconds with approximately 6 total cycles. Additionally, observation of a representative 200 second window (FIG. 7C) in a single channel shows a waveform containing a superposition of several periodic signals. Band pass filtering in the following windows reveals several distinct high-quality signals of interest, ordered in power contribution to the raw signal: (1) 0.01 to 0.25 Hz: a periodic signal with frequency of approximately 3 cycles/min, (2) 0.25 to 5 Hz: a periodic signal with dominant frequency around 26 cycles/min, and (3) Over 5 Hz: a series of repeating spikes with a frequency of approximately 90 cycles per minute. These signals were also observed in separate experiments using the MiGUT system in different animals (N=5, Table 1). Additionally in a separate experiment, euthanization of the animal during MiGUT measurements using a barbiturate cocktail resulted in a cessation of all waveforms (FIG. 7D).

TABLE 1
Slow wave, EKG, and Respiration Amplitudes

| Measurement | Slow wave | Respiration | EKG |
|---|---|---|---|
| # | amplitude (mV) | Amplitude (mV) | Amplitude (mV) |
| 1 | 2.80 ± 1.22 | 0.73 ± 0.17 | 0.06 ± 0.009 |
| 2 | 0.15 ± 0.1 | 0.23 ± 0.04 | 0.013 ± 0.003 |
| 3 | 0.31 ± 0.16 | 0.30 ± 0.04 | 0.08 ± 0.016 |
| 4 | 0.89 ± 0.46 | 0.87 ± 0.04 | 0.078 ± 0.015 |
| 5 | 0.78 ± 0.54 | 0.40 ± 0.03 | 0.063 ± 0.013 |

Due to the signal cessation following euthanization, all of these signals were assessed to be in the various frequency windows described above are of physiologic origin. The observed waveforms with periods of approximately 500 seconds have not been previously observed. The migrating motor complex (MMC) is known to exhibit electrical activity in the pyloric antrum, and has a timescale of 90-120 minutes. The duration of measured signals may correspond to processes at an intermediate timescale between the gastric slow wave and the MMC or are associated with an MMC phase or activity.

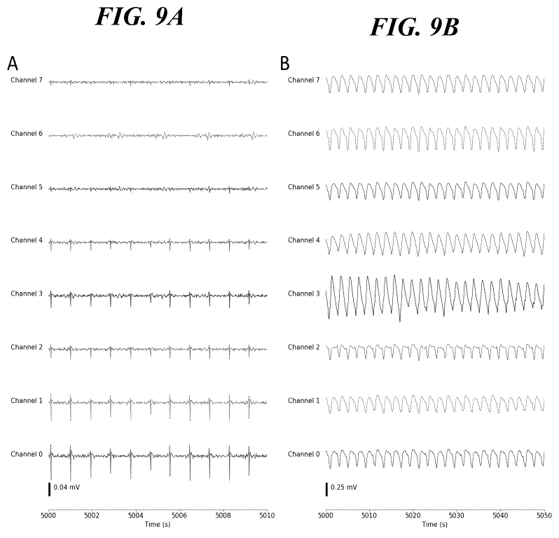

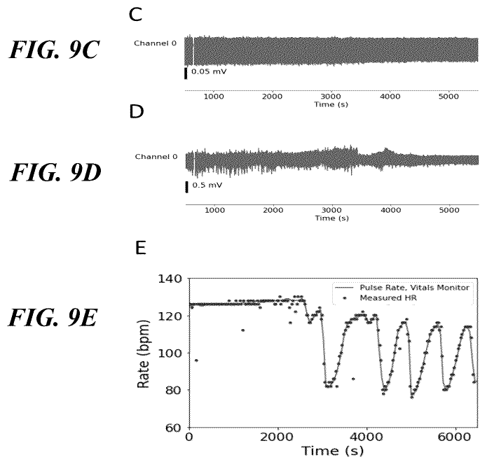

The 3 cycle/min signal is within the range expected for the gastric slow wave generated by interstitial cells of Cajal (ICC), and the shape of the waveform agrees qualitatively with that reported from nasal-clipped gastric mucosal signals. Moreover, this is expected to be the dominant signal in the gastric environment in phases where the slow wave is active. The waveform with frequency of 26 cycles per minute agrees well with the measured respiration rate observed on the vital signs monitor. In addition, the waveform shape agrees with transesophageal diaphragmatic electromyographic measurements conducted using catheter placed electrodes in the esophagus. Finally, the periodic spikes have a frequency of ˜90/minute which agree well with the measured heart rate via the vital monitor and are similar in waveform to an electrocardiogram (EKG) measurement. The MiGUT experimental configuration of reference electrode and electrode ribbon spanning the interior of the stomach is similar to placement of cutaneous electrodes for a standard EKG measurement. Further analysis also shows that EKG signal appears in all channels with the largest amplitude seen in channel 0, corresponding to the channel farthest from the reference electrode (FIG. 9A), and the respiration signal appears in all channels, where amplitude variations between the individual channels are most likely due to the relative location of the electrode/reference and the diaphragm (FIG. 9B). The amplitude of both the EKG and respiration signals remain stable throughout the measurement suggesting that mucosal contact remains reliable for the measurement electrodes (FIGS. 9C and 9D). A separate porcine experiment with a MiGUT device confirms that measured heart rate agrees with measured changes in pulse rate on an external vital signs monitor (FIG. 9E). The observed biopotentials corresponding to the respiration rate and heart rate can be measured through electrodes in the stomach due to the close proximity of the stomach to vital organs including the diaphragm and heart (FIG. 2A). Variability in the detected amplitudes in these frequency windows (Table 1) can be attributed to variations in activity of the stomach, device placement and location of the reference electrode relative to the organ locations.

### MiGUT Measurements Following Prokinetic Agent Delivery

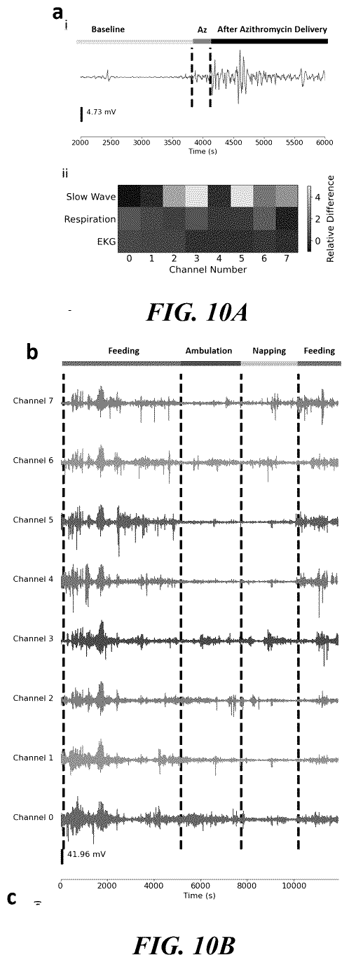

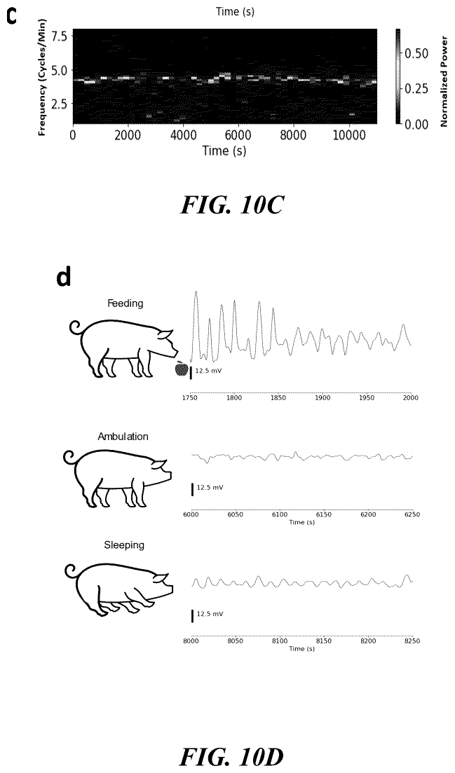

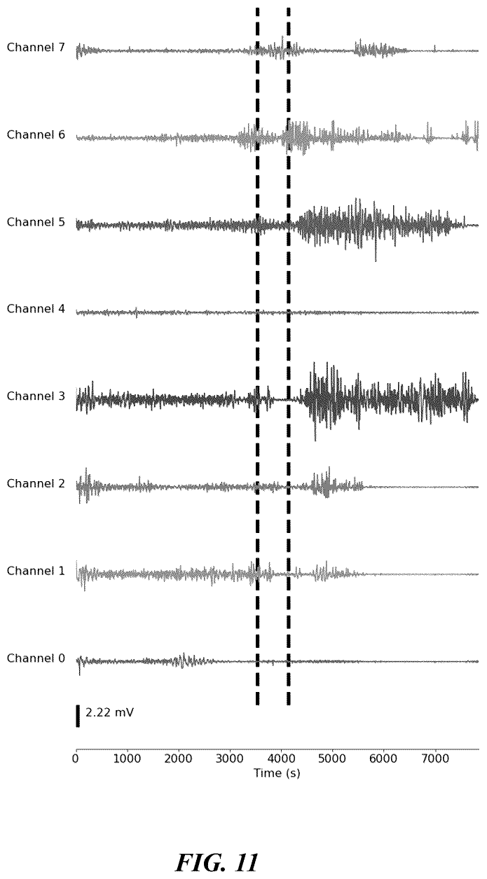

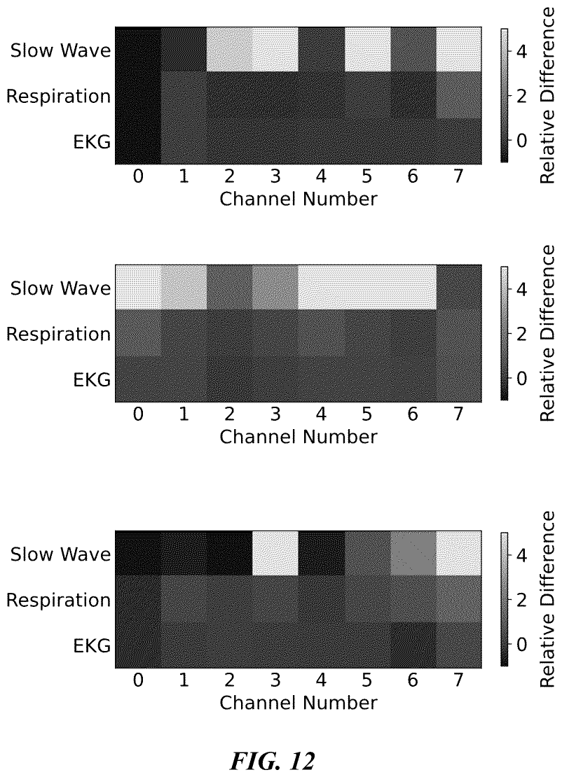

Motivated by the capability of the MiGUT system to detect gastric slow wave activity, an experiment was designed to test if the changes or modulations in gastric motility could also be detected. The MiGUT system was again placed in the porcine stomach of an anesthetized animal and following baseline recording, azithromycin, a known prokinetic which activates motilin receptors, was intravenously delivered (1 mg/kg total dose) via ear catheter over the course of 5 minutes (FIG. 10A(i)). In 5 of the 8 recording channels, a noticeable increase in power was observed over the following 25 minutes in the slow wave frequency range (FIG. 10A(ii), FIG. 11) in comparison to the 25 minutes prior to azithromycin dosing. In contrast, no significant change in power was observed in the EKG or respiratory frequency windows. Replicates of this experiment produced similar increases in the slow wave power (FIG. 12).

The observed increase in power of the slow wave was attributed to the alterations in gastric motility induced from the azithromycin delivery. The power in EKG and respiration windows remain constant indicating electrode contact remains stable following azithromycin delivery (FIG. 10A(ii), FIG. 12). Of additional interest is the noted heterogeneity of the measured response in the different channels which suggests that azithromycin, in addition to induction of increased slow wave activity, may also affect the spatial synchronization of the slow wave conduction along different parts of the GI tract. Qualitatively, this may also be consistent with commonly reported side effects of nausea, indigestion and vomiting following azithromycin administration, possibly as a consequence of desynchronization of the gastric slow wave.

### MiGUT Measurements of Gastric Slow Wave in Freely Moving Large Animal

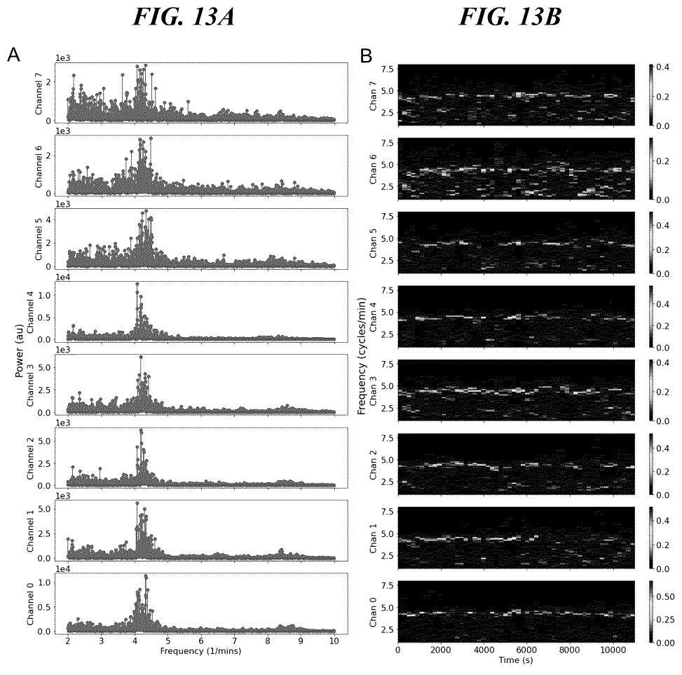

Following experiments of the MiGUT system in anesthetized animals, the potential of the system for monitoring gastric electrophysiology in freely moving and feeding animals was explored. The MiGUT system was delivered to the stomach of an anesthetized swine and secured using endoscopic clips (endoscopic imaging in FIG. 6, schematic in FIG. 8). The animal was allowed to recover in its usual housing and after one hour, timed data recording and transmission was initiated from the MiGUT system for 3.5 hours. Activity in the slow wave frequency window could be measured as the animal went through stages of feeding, ambulation, and napping as indicated (FIG. 10B). Slow wave frequencies in all channels were between 3.5-4 cycles/min throughout the period of measurement (FIG. 10C, FIGS. 13A-13B). The amplitude of the slow waves varied depending on the animal activities, with representative waveforms showing higher 3-6× higher slow wave amplitudes during feeding rather than during sleep or ambulation (FIG. 10D) as noted in representative data segments in a single representative channel.

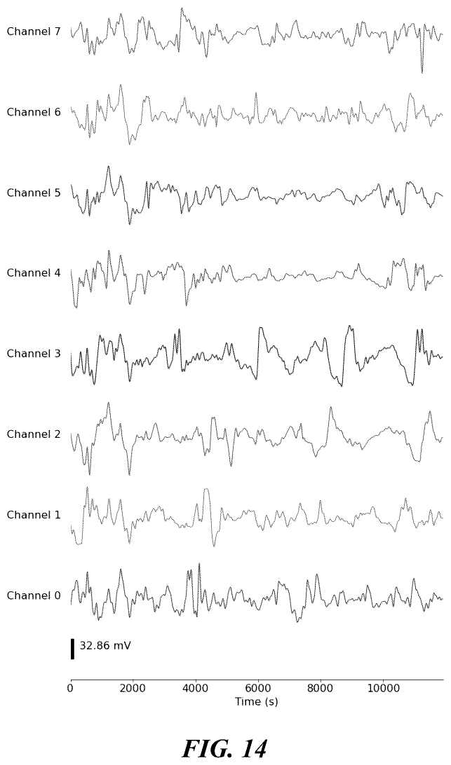

The slow wave amplitudes recorded during animal ambulation and feeding were in the range of 5 mV to 35 mV, which are significantly higher than that observed in the anesthetized measurements (0.5-4 mV, FIG. 7C, Table 1), as well as those of cutaneous measurements (˜50-200 uV) previously reported. The observed difference in amplitudes suggests anesthesia significantly suppresses gastric electrical activity. Additionally of interest, there appear to be minimal mechanical artifacts observed during animal ambulation, suggesting MiGUT can stably measure the gastric slow wave during periods of heightened activities. The distinct increase in amplitude of slow wave activity during feeding and frequency stability of the slow wave agrees well with cutaneous wearable electrogastrogram devices. Following feeding, the slow wave can still be stably measured, indicating that presence of food in the stomach does not inhibit device recordings. Finally, during these freely moving experiments, long period putatively MMC related fluctuations were again observed in a subset of the channels which may be of interest for future investigations (FIG. 14).

### Summary and Outlook

The ingestible MiGUT system is capable of recording high quality electrical signals from the gastric environment, including gastric slow wave, heart rate, respiration rate, as well as long period waves which may be associated with processes related to the migrating myoelectric complex. The MiGUT device demonstrates a concept for monitoring of vital organ monitoring without requiring devices worn/implanted on the skin surface, which could be detached during subject activity. Moreover, medication-induced motility changes were distinguished and the measurement high quality mucosal gastric slow waves during animal feeding and ambulation absent any tethering or wearable system was possible, a capability which has not been previously reported. These results show the potential for long term study of gastric electrophysiology, and a strategy to obtain high quality recordings without restriction to subject movement, meals or day-to-day life.

Further developments to improve the MiGUT system include addition of self-anchoring/resident capabilities and wireless charging, along with increasing channel count via multiplexing, and onboard data filtering/processing. Moreover, there is potential for integration with mucoadhesive/tissue-adhesive systems to further enhance signal fidelity, and electrical stimulation systems to facilitate development of novel closed-loop electroceuticals which reside in the GI tract. This system could study hunger, satiety, and circadian rhythms in the context of chronic GI disorders to understand the fundamental changes in electrical signaling with onset of diseases such as gastroparesis or functional dyspepsia, providing more effective diagnostic and therapeutic targets. The MiGUT may also be used to expand the fundamental understanding of the bioelectric processes of the GI tract and ENS, as well as serve as a valuable tool for diagnosis and management of chronic motility disorders.

### Methods

### MiGUT Electrodes

Three flexible PCB electrodes were developed for use in experiments with MiGUT (FIG. 4). All were manufactured on 75 um thick polyimide with gold plated 0.5 oz copper traces and a 0.5 mm pitch FFC compatible interface on one end. The “large” electrode used double sided, 5 mm diameter measurement electrodes with a 29.44 mm pitch, followed by a 8 mm reference electrode at the furthest end. Another configuration was also manufactured with the reference electrode closest to the FFC interface for convenient device placement. The “medium” electrode was similar in size, but used 0.8 mm diameter measurement electrodes with the same reference electrode configuration and spacing as the “large” electrode. The “small” electrode used 0.8 mm diameter electrodes with 3 mm pitch, followed by a 8 mm reference electrode. Unless otherwise specified, the “large” electrodes were used in experiments presented. To be compatible with Boston Scientific Resolution Clips for experiments with retention, 0.6 mm diameter holes were added to the end of the electrode with stiffener for mechanical support. This allowed ˜10 mm diameter Nitinol wire (NiTi Type 8, 0.025 mm, Fort Wayne Metals) loops to be attached to the electrode ribbon, which were secured to the mucosa using the Resolution Clips.

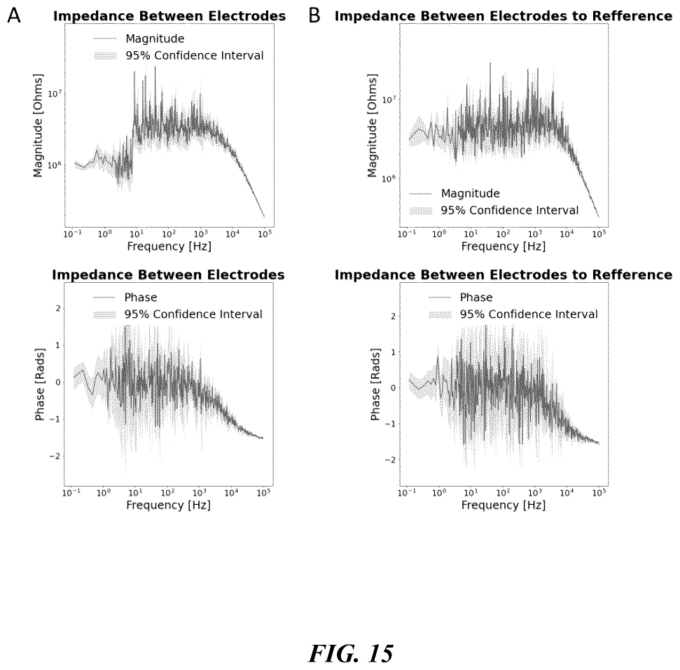

To validate there was no cross talk between the channels, an impedance analyzer (Sciospec ISX-3) was used to determine the electrode-to-electrode impedance. Representative two wire measurements between neighboring electrodes and electrodes to the reference are shown in FIG. 15. The high magnitude of the results indicates there is no cross talk between electrodes, particularly at the low frequencies of interest.

### MiGUT Electronics

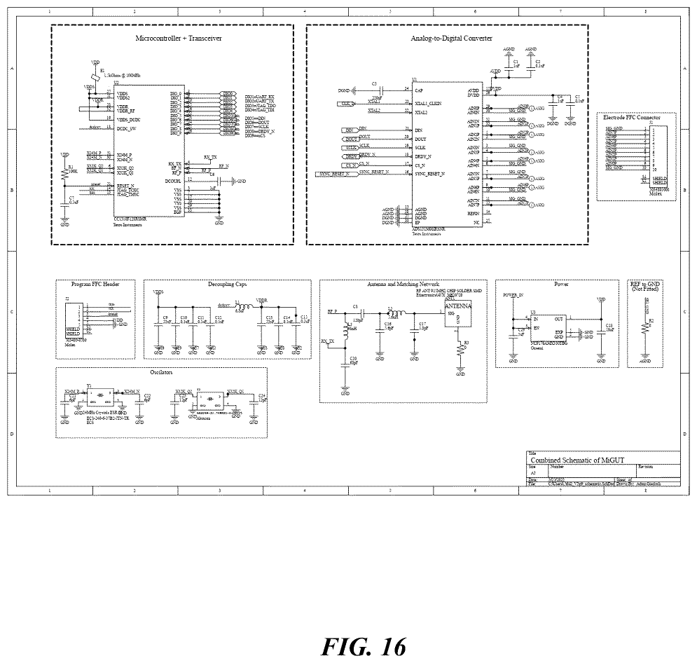

A six-layer Printed Circuit Board (PCB) measuring 7×19.6 mm2 was manufactured using 1.6 mm FR4 substrate, 1 Oz copper traces, and ENIG pad finish. The Flat Flexible Connectors (FFC; Molex 5034801000), Analog-to-Digital Converter (ADC; Texas Instruments ADS131M08), microcontroller with integrated transceiver (Texas Instruments CC1310), crystals (ECS ECS-240-6-37B2-JTN-TR and Abracon ABS04 W-32.768 KHZ-6-B2-T5), and power regulator (Onsemi NCP170AMX330TBG) were then assembled with leadless solder. The schematic illustrating connections is shown in FIG. 16. To provide power, a two-layer daughterboard measuring 7.16×22.46×0.51 mm3 securely mounts the 22 mAh Li-ion battery (Panasonic CG-420) and is connected to the six-layer PCB with headers that are soldered in place. A Li-ion battery was chosen to support the continuous high current draw during wireless streaming of data. The 915 MHz antenna (Ethertronics/AVX M620720) is positioned on the outside face of the assembly to maximize radiation efficiency with recommended keep out regions, and a pi matching network was implemented with datasheet recommended values. Impedance analysis using a Vector Network Analyzer (Keysight E5080B) showed the antenna was not matched, but still showed acceptable performance in-vivo (FIG. 3E).

An off the shelf, eight channel, simultaneous ADC is used to digitize biopotentials. All channels share the same reference, which are connected to the reference pin of the FFC header. The ADC can digitize all channels at sampling rates of 62.5 Hz to 8 kHz when provided with a 2.048 MHz clock, with 24- or 16-bit precision. For space efficiency the clock signal is provided by the microcontroller at 2 MHz, and the desired sampling rates were confirmed to be maintained by injecting a signal at various frequencies and comparing the measured signal.

### MiGUT Configuration

The Cortex-M3 microcontroller is programmed to first initialize the ADC for low power, 16 bit mode, at a programmed sampling rate and signal amplifier gain configuration. For the in-vivo experiments presented, a sampling rate of 62.5SPS was selected for power efficiency and a range of +/−600 mV or +/−150 mV was selected depending on the length of experiment to account for drift. To reduce power, the microcontroller only initializes critical modules and shuts down peripherals used during debugging. The device transmits confirmation that initialization was complete and then transitions to an ultra low power mode for a programmed amount of time. Two programmable arrays allow for complete customizability in sequential lengths of sleep and number of samples taken before transitioning to the other state. Unless otherwise specified, for ambulation experiments the device is turned on immediately before placement, sleeps for 1.5 hours, then records 168750 samples (for 45 minutes, to capture long trends) and sleeps for 22.5 hours (a 2% duty cycle ratio). During sampling, the ADC triggers an interrupt when new data is ready, which is then loaded into a buffer that transmits the data in parallel to receiving new data. For low sampling rates, the device is automatically able to go into a lower power state after transmission while it is waiting for the next sample. The data is wirelessly transmitted at 14 dBm Tx power using 500 kBps symbol rate at 915 MHz with a deviation of 175 kHz and RX Filter bandwidth of 1242 kHz. Data from all channels are transmitted with a packet number and checksumCRC for error correction as they arrive from the ADC.

To evaluate the tradeoff between data throughput and power consumption, the current consumption of the device is measured using the Nordic PPK under different device configurations. When configured to sample at 62.5 samples per second and stream the data continuously, the MiGUT system consumed an average of 6.28 mA with peaks at 21.5 mA. During sleep mode the MiGUT system consumed 50 uA on average. The results when changing the sampling rate are shown in FIG. 3D.

### Receiver Board Configuration

A Texas Instruments LAUNCHXL-CC1310 evaluation board with panel antenna (TE Connectivity PAL902010-FNF) was connected to a laptop and used with Texas Instruments SmartRF Studio 7 (version 2.23.0) to save the data to a text file. While a benchtop receiver setup was used, commercially available USB dongles such as the Texas Instruments CC1111EMK868-915 facilitate data to be recorded to a mobile device.

### MiGUT Device Assembly

The 3D device case was fabricated using a Formlabs 2 3D printer (Durable Resin) in 3 pieces: the main capsule body and two press-fit case caps, one of which has a designed opening for the electrode ribbon to pass through. The PCB was placed into the printed case, the electrode ribbon is connected to the FCC connector, passed through the in the case cap and then both ends of the case closed using the printed caps. The entire capsule was then sealed with UV cure epoxy (loctite 4305) using a UV lamp. The other end of the electrode ribbon is bound to a 3d printed circular roller (9 mm diameter) using water soluble adhesive tape (SmartSolve) and the electrode is rolled up onto the roller, and fixed against the case using the same water soluble tape. For endoscopic clipping experiments, nylon loops are passed to predesigned mounting points in the case, and a nitinol loop is passed through the via/stiffened hole on the double sided reference electrode.

### In-Vivo Testing

All animal experiments were approved by and performed in accordance with the Committee on Animal Care at MIT. Yorkshire swine were obtained from Cummings School of Veterinary Medicine at Tufts University (Grafton, MA, USA) for in-vivo experiments. Pigs weighing approximately 60-100 kg were placed on a liquid diet for 24 hours the day before the study and were fasted overnight prior to the study. For device placement, animals were anesthetized using an intramuscular injection of midazolam 0.25 mg/kg and dexmedetomidine 0.03 mg/kg. Following sedation, animals were placed on thermal support and ophthalmic ointment was applied to both eyes. The animal was intubated and placed on isoflurane (2%) in oxygen and connected to a vital signs monitoring system. The MiGUT device was delivered orally into the stomach, using an orogastric tube and imaged using PENTAX EC-3870TLK (160 cm) to visualize the stomach with the animal in the left lateral position.

### Animal Euthanasia Study

For non-survival procedures, the animal was sedated with intramuscular injection of Telazol (tiletamine/zolazepam) 5 mg/kg and xylazine 2 mg/kg and euthanized using Fatal Plus (sodium pentobarbital), 1 ml/10 lbs. Heart rate and respiration rate are assessed via vitals monitor to ensure that the pig has been euthanized.

### Gastric Transit Studies

For gastric transit studies a device with the same form-factor but enhanced X-ray contrast was used. The interior compartment of the MiGUT device was filled with stainless steel powder (Sigma-Aldrich) to a comparable weight of 3.5-4 g instead of housing the electronics to allow higher contrast under X-ray imaging (FIG. 5). The device was delivered to the anesthetized animal orally through an overtube, and then endoscopically imaged to confirm unrolling in the stomach. All devices (N=3) were radiographically confirmed as excreted by day 5, and were recovered in feces. One device was observed to have a broken ribbon, which was assessed to have occurred following excretion, due to animal activity.

### Azithromycin Motility Experiments Measurements

For azithromycin experiments, following ca. 1 hr of baseline recording in the anesthetized animal, 1 mg/kg animal weight of 20 mg/ml Azithromycin (Sigma-Aldrich) solution (in PBS pH adjusted with HCL, Thermofischer) was delivered via ear catheter over the course of 5 minutes. Measurements were conducted for a minimum of 45 minutes following drug delivery. Power was calculated by summing the square of the voltage measurements in a 25 minute time window. Difference in power was calculated as (Pᵢₙᵢₜᵢₐₗ-P_{final})/Pᵢₙᵢₜᵢₐₗ.

### Ambulating Measurements

Electrodes were fixed to the mucosa following oral delivery using 3 resolution endoclips (Boston Scientific). Two endoclips were placed on the ends of the electronics capsule body and the third on a nitinol loop attached to the reference electrode. Following device delivery and clipping, the swine was allowed to recover for at least 1 hour from anesthesia, prior to ambulating MiGUT measurements. During recovery, the pig was closely monitored until extubation. The animals were returned to their pen and dexmedetomidine was reversed via intramuscularly injected, atipamezole, 0.1 mg/kg. During the recovery process, the animal was monitored until it is considered bright, alert, responsive (BAR). The external 915 MHz receiver was either placed on a wheeled cart outside of the animal pen or mounted on the ceiling of the pen, and animals were fed approximately 1-2 hours following recovery. Device was confirmed to be retained in the stomach 48 HRs following clipping via endoscopic imaging.

### Example 2

### Introduction

Functional gastrointestinal (GI) disorders affect more than 40% of people worldwide, which frequently causes debilitating nausea, abdominal pain, and weight loss, with severe disease resulting in poor nutrition and unintentional weight loss. These disorders, which by definition are abnormalities in the absence of any structural or biochemical findings, present as a complex combination of symptoms unique to each patient and can arise idiopathically or as a complication of other disorders. This results in a lengthy diagnostic process trialing different therapeutics until the patients' symptoms are best resolved. More personalized diagnostic tools are required to determine the underlying cause and lead to faster, objective driven diagnosis.

Similar to how doctors commonly use EKGs and EEGs to diagnose patients with electrical dysfunctions in the heart and brain, there are millions of neurons and associated electrically active cells in the GI tract which control motility, nutrient handling, immune responses and more. However, these signals are poorly understood as clinicians and scientists do not have the tools to effectively monitor these slowly oscillating signals deep within the GI tract for long periods of time. While pioneering work has led to important discoveries from acute, highly invasive measurements, there is still much to be learned from long term recordings as the gut changes on a number of timescales; operating over hours to regulate motility, days depending what meals are ingested, and over the course of months depending on diet and the microbiome. Thus, the ideal clinical system would provide high quality recordings for long periods of time while being easy to deployable as a diagnostic test.

### Neural Activity in the Gut

The Enteric Nervous System (ENS) is comprised of 400 to 600 million neurons spread throughout the GI tract and works with the Central Nervous System (CNS) to control the behavior of various gastric regions. While the measurements from the ENS were first seen over a century, advances in acquisition systems and methodologies have only recently facilitated a better understanding of normal and pathological behaviors, with additional interest in understanding the gut-brain axis.

### History

The first set of experiments presenting the gastric slow wave were performed in 1921 by Walter Alvarez by placing electrodes on the abdominal wall of a woman so thin Alvarez could see her gastric contractions around three cycles per minute. Additionally, around this time I. Harrison Tumpeer and R. C. Davis independently also reported of the electrogastrogram (EGG) signals, from different patient populations and using different recording techniques. Throughout the late 1900s, more validation studies were conducted solidifying the existence of these signals at a large scale, with multiple studies building to the conclusion that these signals were unique pacemakers of the gastrointestinal tract which controlled the frequency of peristaltic contractions in the stomach. Since then, advances in electrode technology, signal processing, and study design led by Lammers, O'Grady, Kelly, Du, and more have led to a more complete understanding of the electrical activity in the gut.

### Physiological and Pathological Behaviors

The major components of the GI tract consist of the esophagus, stomach, small and large intestines, and anus, each of which have their own characteristic electrical behaviors. The stomach is the focus of this example as it has been the most well studied and clinically relevant, but the key components are recently starting to be analyzed in the intestines and esophagus. The stomach is responsible for accommodating (growing in size) to accept food, churning the food for easier digestion by means of peristaltic waves (Fed Motor Pattern), and emptying in a series of complex contractions driven by the Migrating Motor Complex (MMC).

Normal behavior in the human stomach revolves around the proper generation of the slow wave, originating from pacemaker Interstitial Cells of Cajal (ICC). Waves in the pacemaker region of humans have been recorded at a frequency of approximately 3 cpm traveling with a velocity of 8 mm/s with an amplitude of 0.57 mV. These waves propagate circumferentially down the corpus at a velocity of ˜3 mm/s, faster in the greater curvature to stay in phase with the lesser curvature, and decrease in amplitude to 0.25 mV. Waves traveling up towards the fundus quickly slow and attenuate. These waves are ever present but below the threshold voltage required to elicit peristaltic waves, which only occur when spiking activity is superimposed on the slow wave to surpass the required threshold voltage. Waves in the antrum are more complex, and rapidly accelerate to 5.7 mm/s with an increased amplitude of 0.52 mV. These results from a fasted patient under anesthesia are supplemented by implanted electrodes showing similar results. In orchestration with feedback networks from other neural pathways, these slow waves and generated spike potentials control the peristaltic waves that break up food and empty the stomach.

Pathological behaviors in the stomach have historically been investigated through the lens of slow wave frequency in mirroring analysis from the heart. Tachygastria is defined as abnormally high frequency of the slow wave (3.7-10 cpm), while an abnormally low frequency corresponds with Bradygastria (0-2.5 cpm). These abnormal slow wave frequencies have been shown to strongly correlate with loss of ICC pacemaker cells [26]. However non-invasive wearable tools for measuring the slow wave frequency have yet to be widely adopted due to limited diagnostic understanding. To go beyond frequency analysis, O'Grady et al. used their high resolution mapping technique to spatially map the stomach of patients with gastroparesis, finding various abnormal initiation and abnormal conduction events. While more research is still required, this supports a more intimate understanding of the stomachs state could better diagnose different gastric neuropathies; similar to how single channel EKGs are acceptable for fitness trackers to identify when something is wrong, and diagnostics typically requires multiple channel EKGs.

### The Gut-Brain Axis

In addition to acute measurements of the pacemaker region and slow waves, fundamental research around the gut-brain axis would benefit greatly from long term measurements. This connection with the brain has been reported in patients with Parkinson's disease who present with GI motility issues before cognitive symptoms arise, and many with autism spectrum disorder present with gastrointestinal issues. Biopotential measurements tracking organ level changes may eventually be used to infer changes in the microbiome and other but-brain interactions.

### Medical Devices for Gastric Recording

A number of new systems and devices have been created to facilitate the study of biopotentials in the GI tract. These can be subdivided into tethered, implantable, and wearable devices.

### Tether Systems

Tethered systems involve directly connecting the recording electrodes to an external acquisition system using long cables. By removing any size requirements from the recording device, this has facilitated a number of high channel count, high resolution studies to be conducted. Two standout systems are those used by Lammers and O'Grady. Lammers et al. were the first to do high resolution mapping and recorded 240 channels using an 8-bit 1 kSPS recording system designed for recording myocardial signals, connected to custom fabricated silver wire electrodes (0.3 mm diameter in a 15×16 grid with 1 mm spacing) in rabbit. Inspired by this, O'Grady et al. recorded 192 channels in human using an ActiveTwo System (Biosemi, Amsterdam, The Netherlands) sampling at 512 SPS, with multiple gold electrodes in a 4×8 array spaced 7.6 mm apart. The gold electrodes were compared to Lammers silver wire electrodes and found to give comparable results. Through these systems, the origin and propagation of the gastric slow wave has been thoroughly described in the healthy and pathologic human stomach, and throughout large animal models.

However, these systems come at the cost of invasiveness and provide only acute recordings during an operation. In some such systems, patients volunteered for this study as they underwent elective surgery, but it is unlikely an operation could be as a standard diagnostic test. Additionally, while in the aforementioned studies in this example great care was taken to ensure minimal movement of the organs, it has been shown that movement can cause abnormal behaviors likely due to activating stretch receptors. The operating room setting may have also resulted in abnormal recordings due to the surgery preparations. Specifically, as patients fast prior to surgery, behaviors such as the migrating motor complex and peristaltic waves may be altered. Lastly, there are conflicting viewpoints on the effect of anesthesia, with the majority agreeing it slightly to significantly diminishes the slow wave. Overall, more data is still required to comprehensively address the impact of these limitations.

To overcome these challenges, Angeli et al. developed a high resolution mapping device that can record from the mucosa using endoscopic intubation. This used the Constellation™ mapping catheter (Boston Scientific, Marlborough, MA, USA) to record from eight rings of eight electrodes spaced 7 mm apart, and the same acquisition system as previously used by O'Grady. This study highlighted that recordings from the mucosa are comparable to those recorded from the serosa. Other groups have since shown mucosal recordings from ambulating patients, but these are limited in the number of electrodes and required nasally clipped leads passed up the esophagus. While in the right direction, these tethered systems do not allow the patient to go about their daily lives for long periods of time.

### Implantable Devices

To gather data before and after eating, implantable devices have been developed to record biopotentials over lover periods of time. In humans, the majority of data comes from Gastric Electrical Stimulator (GES) devices with recording channels. For power efficiency, these devices typically sample at much slower rates with fewer channels; in a previous study, samples were recorded at 60 SPS using a 12 bit analog to digital converter from four channels. These studies have facilitated recordings to be taken over multiple days while patients go about their daily lives but require invasive surgical placement. Other devices by have also been developed, but these devices are quite large and have other tradeoffs (further described later herein).

### Wearable Devices

Since the discovery by Alvarez using surface electrodes, the quality of electrogastrography (EGG) recordings have significantly improved but have yet to find their place in the standard diagnostic procedure. The key challenge of these wearable systems is recording the heavily attenuated signal (by the abdomen) and minimizing artifacts from respiration and motion. A number of signal processing techniques have been developed to recover the low amplitude slow waves while also removing motion artifacts, but the best recordings still come from immobilized patients. To assist in these efforts, new multi-channel EGG systems have been shown to reveal the slow wave direction and speed in addition to frequency when coupled with advanced signal processing. However the quality of the recorded data still prevents widespread adoption.

### Development of an Ingestible Device for High Quality Neural Recordings

Here the development of an ingestible device for Multimodal electrophysiology via Ingestible, Gastric, Untethered Tracking (MiGUT) is presented. First the design considerations are presented, followed by the development of conformal electrodes for neural recording in the stomach, then the design and validation of low power, wireless electronics.

### Design Considerations

### Opportunities for Ingestible Devices

Ingestible devices offer a noninvasive way to access signals deep within the body from the gastrointestinal tract. Endoscopic techniques are an important tool for visually evaluating the GI tract, with new endoscopic tools facilitating more advanced tests and procedures such as photoacoustic and ultrasound imaging, laser therapy, and more. Ingestible devices could be designed to achieve the same level of biomarker tracking and/or therapy while also being less invasive than endoscopy (typically requiring sedation). This has been proven in devices such as the FDA Cleared Medtronic PillCam which provides good visualization of the colon without the drawbacks associated with endoscopy. Since this first adoption and acceptance of ingestible devices clinically, other ingestible devices have been designed with different sensing modalities such as pH, temperature, pressure, bacterial and gas sensing, ultrasonic imaging, and modulation devices. In addition to studying the gastrointestinal tract, ingestible devices boarder or transit within close proximity of other key organs, which could provide an effective avenue for high fidelity measurements or targeted therapies. This could facilitate a no instructions needed diagnostic tools to be easily deployed in or shipped to each person's home someday.

### Key Challenges

There are a few fundamental challenges and constraints to consider when designing ingestible, especially relating to size, communication, power, and interface.

One challenge with ingestible devices is size. The goal is to pack as much smarts and power into the device while allowing it to still be safely ingested and pass through the GI tract. Currently the largest standard pill size used is the 000 (“triple zero”) capsule with a diameter of 9.91 mm and a length of 26 mm. However, these gelatin capsules usually dissolve to release their therapeutic. Passage studies of various capsule sizes show in order to ensure a there is minimal risk of blockage, which constitutes a life threatening complication, a device should have its longest dimension be no more than 15.5 mm. This is a challenge with ingestible devices, which typically carry onboard batteries for power, large antennas, and electronic circuitry; the PillCam which measures ø11×26 mm is retained at a rate of 1.4%. While no studies have been done on device roughness, it is generally desirable for devices to be smooth and not cut inner tissues. However, case studies have revealed ingestion of razor blades can pass safely without intervention, indicating deviations from standard pill shapes more accommodating to packing dense electronics may be acceptable.

Closely related to size is power, specifically power density. While implantable devices such as neurostimulators or pace makers use lithium-based batteries, these are very large and not amenable to an ingestible form factor. Most ingestible on the market use Silver-Oxide (AgO2) batteries for their safety and typically contain higher power density than lithium-ion batteries. However, AgO2 batteries are not typically suited to drive the high current consumption required for wireless communication. Lithium-ion batteries are convenient for their ability to be recharged and provide high levels of sustained current but are typically considered too volatile if there is a concern of gastric fluid leakage into the device. Battery packaging also needs to be considered for overall size optimization. While this is an active area of research, future devices will likely rely on wireless power solutions or wireless charging to ensure long term use.

Wireless communication methods for implantable and ingestible devices include piezo electric (ultrasound), radio frequency, optical, and magnetic modalities. However the environment of the GI tract is much deeper, with more diverse tissue layers between the device and the receiver. This creates issues relating to penetration depth of different radio wave frequencies, while other communication methods typically require bulky external receivers. Current ingestible devices compromise by using sub 1-GHz radio frequencies, specifically in the ISM band (433 MHz or 915 MHz), although recent devices have been shown to successfully implement Bluetooth (2.4 GHz) communication with careful antenna tuning.

Finally a stable interface is required to make the device meaningful. Rigid electronics and electrodes limit their size as the gastrointestinal tract consists of many folds and is in motion. Current tethered devices use flexible electrodes, but these have only been extensively tested on the serosa. Electrodes for interfacing with the mucosa have typically been individual wire electrodes secured to the mucosa with clips or pressed against the mucosa using a balloon. Additionally, the electrodes should be of inert material which does not react with the gastric environment that has a wide pH range (pH of 2.1 in the fasted state or 3-7 while fed in the stomach, which can be different in the upper and lower stomach, and increasing pH in the intestines) and contains many other harsh digestive compounds.

### Design Decisions

Taking into account the above challenges and current implementations for mitigating them, the following initial design decisions were formed: PillCam or smaller device dimensions to ensure a future path for clinical translation should be met. Specifically, a maximum diameter of 11 mm and length of 26 mm or smaller, allowing for flexibility in using off the shelf components. Further miniaturization, specifically such that the maximum dimension is under 15.5 mm, will be outlined in later herein.

Li-ion battery power will be used for flexibility in device design and ease of use for later data gathering stages (allowing for recharging and reuse of the device while providing a path for wireless charging to increase recording length). Powering with Silver Oxide batteries must be demonstrated to ensure safety of future devices but does not have to be implemented in this version.

915 MHz communication will be used as it has been proven as a robust communication method in commercially used ingestible.

Custom flexible electrodes will be designed described later herein.

### Conformal Electrodes for Neural Recordings

To acquire neural signals from the gastrointestinal tract, a number of strategies have been taken. While wireless measurements using Magnetic Resonance Imaging (MRI) and optogenetics have been previously reported, the best results come from metal electrodes measuring the electrical signals directly. MRI is limited to measuring contractions and has resolution limits impeding study of complex behaviors, while optogenetics are not translatable to humans. In contrast, metal electrodes can be used for long term recordings and have been used in many pacemakers and neurostimulators with minimal complications. The current challenges with metal electrodes revolve around maintaining a stable interface to ensure high quality recordings in the environment of the GI tract.

### Micron Thick Conformal Electrodes

In comparing current electrode designs, a multi electrode array arrangement is seen to be the most valuable arrangement—facilitating the analysis of slow wave direction and velocity. However the current solutions, even ones made from flexible substrates, still remain relatively stiff as the size grows to accommodate a larger grid of electrodes. In experiments, wet gauze to be placed over the electrodes is commonly used to ensure good contact (in addition to ensuring the interface does not dry). By removing the thickness in the z-dimension, it was expected that the large electrode arrays could more effectively conform to the surface of the mucosa.

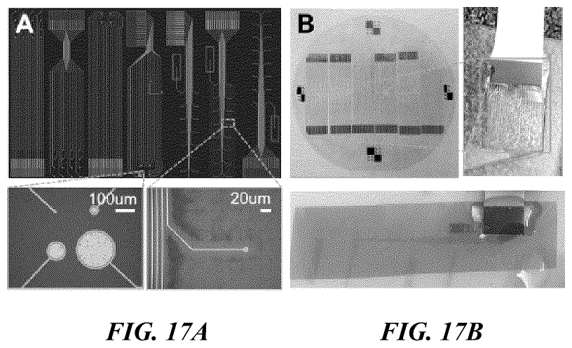

Multiple sixteen-channel electrodes were designed as shown in FIG. 17, with varying degrees of electrode size (5 um to 200 um diameter) and electrode spacing (pitch spanning 200 um to 4 mm). Specifically, to test the impact of electrode size one design incorporated four electrodes with exposed pads of diameter 5 um, 30 um, 80 um and 180 um (cut out 10 um smaller than actual electrode size on all sides for mechanical stability) with a pitch of 200 um (FIG. 17A). Another small electrode design fabricated was inspired from neural multi electrode arrays was a 4×4 grid of 30 um diameter electrodes with a pitch of 200 um (Multichannel Systems GmbH, BW, Germany). Most other electrodes were designed to sweep the pitch from 1 mm to 4 mm with varying grid arrangements, since the slow wave spans much greater distances. To interface with different recording systems, contacts were placed with 0.5 mm pitch to allow for mating with FFC connectors/cables. To assist with the flexibility, it was hypothesized adding 20 um diameter holes with a pitch of 100 um to 200 um would allow for additional flexibility and were added to some designs (FIG. 17A). Additionally, some designs included a pivot point and/or ninety-degree rotation from the connector side to decouple movements from the connector side.

The fabricated electrodes were manufactured in the Harvard Center for Nanoscale Systems [a member of the National Nanotechnology Coordinated Infrastructure Network (NNCI)] on three inch silicon wafers (Nova Materials, CP02-11208-OX), in a protocol similar to. The wafer was first cleaned with O2 plasma (100 W, 2 min, 50 sccm), and a ˜500 nm layer of polyamide applied with a spin coater at 500 rpm for 5 seconds followed by 3 k rpm for 45 seconds. The wafer was then soft baked for 4 minutes at 65° C. followed by 2 hours at 200° C., and cooled at approximately 5° C./min to prevent cracking. This forms the bottom layer insulating layer to isolate the electrodes. Next, photoresist (LOR3A) was spin coated (500 rpm for 5 s, then 3 k rpm for 45 s) followed a soft bake for 3 min at 115° C. and S1805 photoresist added in a similar fashion. The electrode designs were then exposed onto the wafer using a Heidelberg MLA150. After the wafer was descummed (O2 plasma at 30 W, 30 sec, 50 sccm), 5 nm of titanium followed by 100 nm of gold was deposited using an electron beam evaporator. After deposition, the wafers were placed in acetone filled containers and placed on shakers for two hours for lift off, revealing the electrode designs. To add the top insulating layer of polyamide such that the traces were not exposed to the environment, an additional layer of polyamide approximately 500 nm thick was deposited as before. To etch out the electrode pads, S1822 photoresist was spun onto the wafers (same settings), followed by soft bake at 115° C. for 5 min, and then exposed using the maskless aligner as before after alignment. Finally, the windows were etched using the RIE8 followed by washing in CD-26 for four minutes to remove any remaining photoresist. This resulted in approximately 1 um thick electrodes of multiple designs, with quality of windows and perforations shown in FIG. 17A cutouts.

To release the electrodes from the wafer, water soluble transfer tape (SmartSolve, OH, USA) was placed on top of the wafer, the wafer was cracked from the other side, and the wafer was peeled away. This left the electrodes stuck to the water soluble tape, with electrodes facing inwards (FIG. 17B). The electrodes were then released onto Kapton tape or a layer of Poly Lactic-co-Glycolic Acid (PLGA) polymer by carefully washing away the water soluble tape, leaving the electrodes outwardly facing and temporarily supported so as to not curl. Following this, a 16 channel flat flexible cable was pressed onto the electrode pads with anisotropic conducting film (3M 7303) to provide a secure electrical connection. Finally the interface was covered with epoxy to ensure a robust connection, with the end result shown in FIG. 17B.

In-vivo measurements with Commercial Recording System

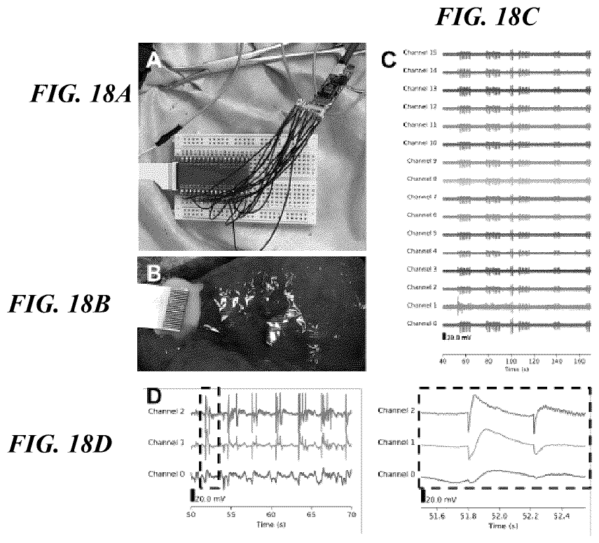

To determine a baseline of expected electrophysiology measurements from the stomach and evaluate these custom electrodes, an adapter board was made to connect the electrodes to a commercially available benchtop recording system. The Open-Ephys (open-ephys.org) Acquisition Board with Intan (CA, USA) RHD2132 headstage connected to the electrodes via FFC cable was used to record biopotentials at 30 k samples per second with a resolution of 16 bits and a dynamic range of 15 mV (FIG. 18A). This system was implemented during an in-vivo study in a sedated Yorkshire Pig, with all animal experiments approved by and performed in accordance with the Committee on Animal Care at MIT. After sedation, access to the mucosa of the stomach was gained through laparotomy and the custom electrodes were removed from their Kapton support and placed directly on the mucosa (FIG. 18B).

The electrodes were seen to conform well, with contact verified by impedance measurements. With an AgCl electrode placed in at the bottom of the stomach, electrode impedances in the range of 0.18 MOhm to 9.64 MOhms were observed (a decrease of ˜10 MOhm compared to baseline). Recordings were seen in all channels with sets of spike potentials seen at a frequency of around three cycles per minute (FIGS. 18C-18D), matching well with the expected frequency of the slow wave. However due to the all the adapter boards adding parasitic capacitance combined with a high pass filter frequency around 1 Hz, the slow wave (around 0.05 Hz) was not recorded. Evaluation of these electrodes is left as future work with MiGUT.

### Flexible PCB Electrodes

To facilitate more rapid prototyping and a higher throughput of experiments, gold flexible electrodes were developed for the following experiments using commercial flexible printed circuit board manufacturing. With a focus on long term studies, recording from multiple areas of the stomach instead of just one localized region was desirable. As such, long flexible electrodes of 25 cm in length that could conform to the greater curvature of the pigs' stomach were designed as shown in FIG. 4. The low cost of these electrodes facilitated multiple designs, varying the electrode size and pitch for different applications. Importantly, stiffener is applied to the connection end with a pitch of 0.5 mm allowing for integration into the same FFC connectors as the previously designed electrodes. Additionally, these electrodes can be easily rolled up on a 9 mm roller and secured with water soluble tape for easy deployment into the stomach (FIGS. 2C-2D).

To ensure no cross talk between electrodes, two point impedance measurements were performed using a Sciospec ISX-3 Impedance Analyzer (Bennewitz, Germany). FIG. 15 shows high impedance between two adjacent electrodes and a high impedance between each electrode and the reference electrode. This suggests there is enough isolation between each channel to ensure independent measurements.

While not tested using the benchtop system, these electrodes were incorporated with MiGUT to achieve high quality results in-vivo.

### Low Power, Wireless Electronics

### Initial Prototype

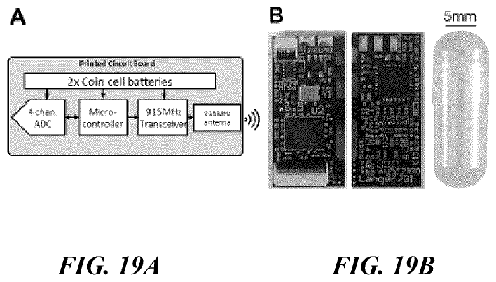

First, an initial prototype was developed to show all the necessary components could be integrated into a custom printed circuit board in an ingestible formfactor. A six layer PCB was designed to sample data at 215 samples per second per channel using a 16 bit four channel analog to digital converter (ADC; Texas Instruments ASD1115), send the data to a microcontroller for data handling (Analog Devices ADuCM355), and wirelessly transmit the data using a 915 MHz transceiver (Semtech SX1231). This system, facilitated by the ADuCM335, could also perform impedance measurements and cyclic voltammetry to test other ingestible sensors. All of this was integrated into a 25×10 mm2 PCB shown in FIGS. 19A-19B, which could be powered by size 13 hearing aid coin cell batteries (Duracell). However the current draw of the device peaked at 112 mW during transmission significantly reducing the battery life, even when heavily duty cycled. While only four channels were bipolar on the dedicated ADC, eight additional ADC inputs along with the two chemical sensing channels were made available from the microcontroller with an additional flat flexible connector. Thus, all necessary components were successfully shown to be further miniaturized from the current state of the art ingestible devices (compared elsewhere herein), a key step to facilitate clinical translation of this system.

### MiGUT Overview

After developing the first iteration, the final device was designed to improve the size, battery life, and ease of assembly. MiGUT is shown in FIG. 3A which consists of an electronics module with a daughter board providing power, and can connect to a number of flexible measurement electrodes via a flat flexible connector. The electronics are housed in a 9×12×26 mm3 3D printed case (FIG. 3B) which was designed to be comparable to DFA cleared products such as the capsule endoscopy systems (Medtronic PillCam, ø11×26 mm3) to facilitate future clinical translation.

With a focus on miniaturizing MiGUT even further compared to the initial prototype, the Texas Instruments CC1310 microcontroller with integrated transceiver was selected to control the sensing peripherals and handle wireless communication. The 4×4 mm2 package was used for the CC1310 which is smaller than the initial versions microcontroller alone, at the cost of the TIA, impedance measurements, and digital waveform generators present in the ADuCM355—valuable for other experiments but not critical for the design. This facilitated the incorporation of the Texas Instruments ADS131M08, a more versatile eight channel 24 bit simultaneous ADC. This ADC was selected for its ability to record bipolar potentials simultaneously at configurable data rates from 65.4 samples per second to 8 k samples per second (in very low power mode).

All channels share the same reference, which are connected to the reference pin of the FFC header. For efficient data movement and wireless communication, the ADC was set to digitize with 16 bits. The updated schematic is shown in FIG. 3C, with the detailed schematic containing passives and power circuitry in FIG. 16.

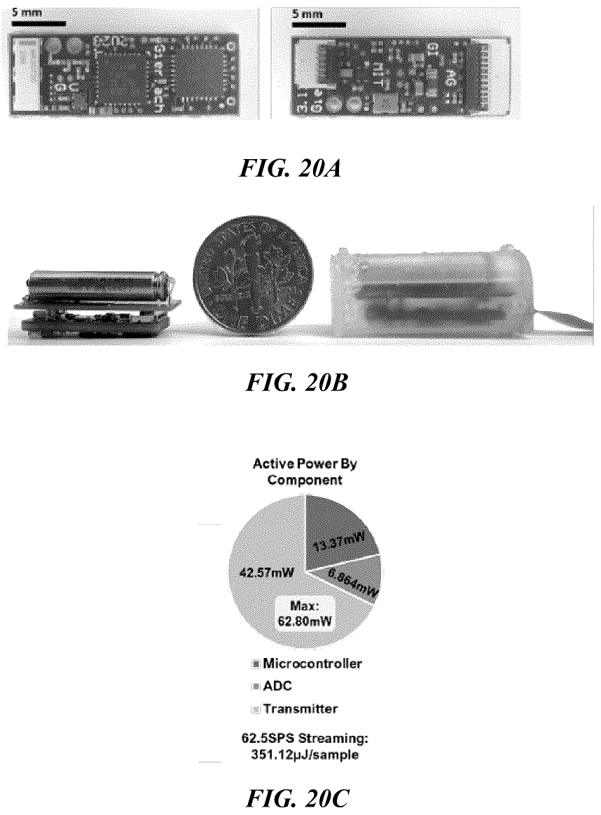

This schematic was realized on a six layer custom printed circuit board measuring 7×19.6 mm2 (FIG. 20A) manufactured using 1.6 mm FR4 substrate, 1 Oz copper traces, and ENIG pad finishes. The Flat Flexible Connectors (FFC; Molex 5034801000), Analog-to-Digital Converter (ADC; Texas Instruments ADS131M08), microcontroller with integrated transceiver (Texas Instruments CC1310), crystals (ECS ECS-240-6-37B2-JTN-TR and Abracon ABS04 W-32.768 KHZ-6-B2-T5), and power regulator (Onsemi NCP170AMX330TBG) were then assembled with leadless solder. To power the device, a two layer daughterboard measuring 7.16×22.46×0.51 mm3 was used to securely mount the 22 mAh Panasonic CG-420A Li-ion battery and is connected to the six layer PCB with headers that are soldered in place (FIG. 20B, left). This facilitates different power configurations to be used in the future depending on the study requirements. A Li-ion battery was chosen to support the continuous high current draw during wireless streaming of data, with more details elsewhere herein. The 915 MHz antenna (Ethertronics/AVX M620720) was positioned on the outside face of the device to maximize radiation efficiency with recommended keep out regions, and a pi matching network was implemented with datasheet recommended values. Impedance analysis using a Vector Network Analyzer (Keysight E5080B) showed the antenna was not matched, but still showed acceptable performance in-vivo as described elsewhere herein.

The device was then placed in a 3D printed case (FIG. 20B, right) fabricated using a Formlabs 2 3D printer (Durable Resin), with end caps press fit together, and sealed with UV cure epoxy (Loctite 4305), bringing the final dimensions to 9×12×26 mm3. The electrode ribbon is connected to the FFC connector before placing into the case, with the electrodes fed through a slot in one of the end caps, and then sealed with the UV epoxy. For experiments where a roller was used, the electrode ribbon was bound to a 9 mm diameter roller using water soluble tape as shown elsewhere herein for ingestible delivery with automatic deployment once the device reaches the stomach. For experiments where endoscopic clipping was used, nylon loops can be passed through mounting points in the 3D printed case at the front and back ends of the capsule. Additionally, a nitinol loop can be passed through the via located on the double-sided reference electrode for secure placement of the flexible electrodes and ensuring a secure interface with tissue.

Further reductions in size could be achieved by using tighter PCB manufacturing tolerances, different battery configurations, or combining all the components into a single custom integrated circuit. Other deployment and retention mechanisms can easily be integrated into the case for ease of deployment and retention.

### Software Flow and Power Management

The current consumption of the device depends on the sampling frequency of the ADC, frequency of wireless communication, and duty cycling rate—all of which can be programmed ahead providing flexibility in balancing experiment length and signal fidelity. First, the Cortex-M3 microcontroller is programmed to initialize the ADC for low power in 16 bit resolution mode, at a specified sampling rate and signal amplifier gain configuration. For the in-vivo experiments presented, a sampling rate of 62.5 samples per second was selected for power efficiency and a range of +/−600 mV or +/−150 mV was selected depending on the length of experiment to account for baseline drift. Then, the device transmits a confirmation it was successfully initialized and then transitions to an ultra low power mode for a programmed amount of time. Two programmable arrays allow for complete customizability in the duty cycle ratio over multiple recording events. Unless otherwise specified, for ambulation experiments the device is turned on immediately before placement (to allow for confirmation of functionality once assembled), sleeps for two hours, then records 168750 samples (for 45 minutes, to capture long trends), and sleeps for 22 hours (a 2% duty cycle ratio). During sampling, the ADC triggers an interrupt when new data is ready, which is then loaded into a buffer that transmits the data immediately in parallel to receiving new data. While schemes for saving the data were explored, streaming the data was found to be more robust in ambulating experiments to ensure minimal interruption of data if one of the packets was corrupted. For low sampling rates, the device is automatically able to go into a lower power state after transmission while it is waiting for the next sample. The data is then wirelessly transmitted as described in elsewhere herein.

To evaluate the trade-off between data throughput and power consumption, the current consumption of the device was measured using the Nordic Semiconductor Power Profiler Kit II under different device configurations. These measurements were validated in long term current consumption measurements using a Keysight 34465A digital multi meter. When configured to sample at 62.5 samples per second and stream the data continuously, MiGUT consumed an average of 6.64 mA with peaks at 16.88 mA. During sleep mode MiGUT consumed 6 uA on average. The results when changing the sampling rate are shown in FIG. 3D.

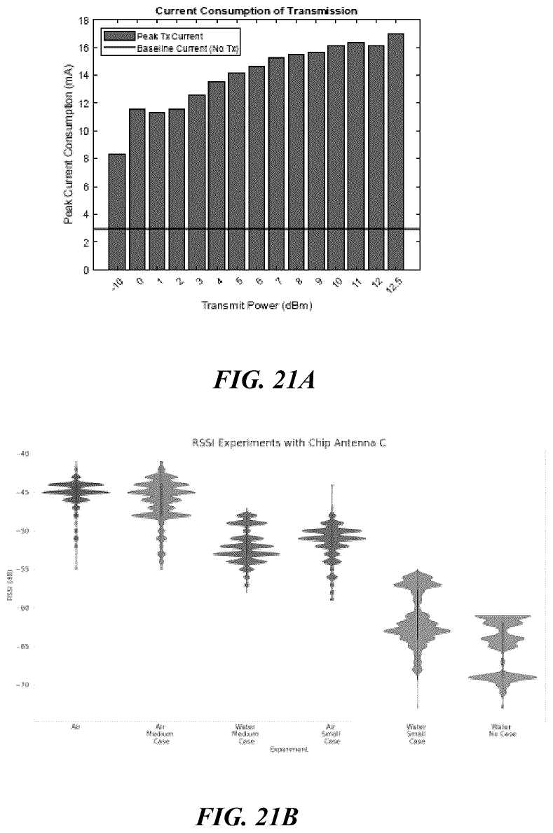

While a Li-ion battery was chosen to ensure the high current peaks during wireless transmission would not damage the battery (FIG. 20C) and allow for more efficient use via recharging, silver oxide batteries are more commonly used in ingestible devices for their safety. Thus, in addition to the Panasonic CG-420 li-ion, two Murata SR626 (1.5V 28 mAh) and two Renata 393 (1.5V 80 mAh) in series were separately tested to show compatibility for future translation. While the two Murata SR626 configuration only lasted approximately five minutes before continually resetting and unable to maintain nominal operation, the two Renata 393 battery configuration was able to last 7 days. This is 3.5× times longer than the Panasonic CG-420, with only a slight size increase of 1.5×, at the cost of being unable to recharge and a cylindrical form factor. In cases where the smaller form factor of the Murata SR626 battery is more desirable, the transmit power of the device can be lowered. FIG. 21A shows the current consumption of only the CC1310 during different transmit power levels. This can be correlated with the datasheet or other stress tests to choose a transmit power level with acceptable current peaks for each battery, such as 9 dBm for the Murata SR626.

Wireless Communication

Data from MiGUT is wirelessly transmitted at 12.5 dBm transmit power using a 500 kBps symbol rate at 915 MHz with a deviation of 175 kHz and receiver filter bandwidth of 1242 kHz.

Data from all channels are transmitted with a packet number and checksum CRC for error correction as they arrive from the ADC, resulting in a packet of 20 bytes plus address and preamble. Generally shorter packets were found to be received more effectively, with longer packets generating more likely to be corrupted during transmission in non-ideal conditions, though this was not thoroughly explored. The 915 MHz communication frequency was chosen for its greater penetration depth and proven track record in ingestible devices.

Tests with Bluetooth Low Energy were also performed contributing to and showed extra considerations are required when trying to implement Bluetooth communication in an ingestible formfactor. FIG. 21B shows the impact of different spacing between an antenna and the case when placed in a 500 mL beaker of water. While this shows the absolute signal strength can be made acceptable, the bit error rate of the signal was found to cause issues when attempting to connect during in-vivo studies. A benefit of 915 MHz is that the packets are consistently transmitted at one frequency (compared to frequency hopping possibly during Bluetooth connections) and does not require a handshake to start communication.

A Texas Instruments LAUNCHXL-CC1310 evaluation board with panel antenna (TE Connectivity PAL902010-FNF) was connected to a laptop and used with Texas Instruments SmartRF Studio 7 (version 2.23.0) to save the received data to a text file. While a benchtop receiver setup was used, commercially available USB dongles such as the Texas Instruments CC1111EMK868-915 can facilitate data to be recorded to a mobile device. With the panel antenna approximately one to four meters away from the device in a freely moving Yorkshire pig (97 kg), an external 915 MHz transceiver was found to reliably receive over 99% of data from MiGUT at approximately −70 dBm in a full stomach and during multiple behaviors (FIG. 21B).

To determine the minimum transmit power required for effective communication, the packets were repeated and transmitted at different transmit power levels in a different 87 kg pig. The results shown in FIG. 3E show that good communication can be maintained from +12.3 dBm to approximately 4 dBm, after which the RSSI drops and the bit error rate increases considerably. Due to the structure of the enclosure, which contains metal walls that could impede wireless signals, +12.5 dBm was used to ensure an adequate safety margin. However in less obstructed environments, if +4 dBm transmit power is acceptable this would correspond to a 1.8× power decrease (FIG. 21A).

### Measurement Validation

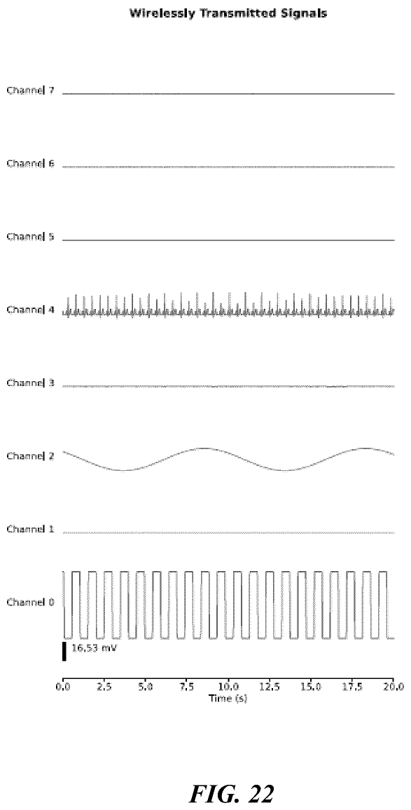

To validate the operation of each device, a known stimulus is injected into the electrodes in addition to power profile measurements. A 1 Hz 200 mV peak to peak square wave generated by a signal generator (Keysight 3000T with DSOX3WAVEGEN or Rigol DS1104Z-S Plus) was measured on all channels individually to confirm sampling rate and correct voltage decoding. Other signals were also tested over a variety of amplitudes and frequencies, and the correct output was seen to be received with no cross talk. One such test is shown in FIG. 22.

### In-Vivo Validation of MiGUT in Anesthetized Animals

In-vivo demonstration of MiGUT was first performed under controlled conditions to ensure adequate signal acquisition, in addition to the performance characterization already shown. While some early experiments with the initial prototype and cleanroom electrodes were performed in a terminal setting with surgical access via laparotomy, all experiments shown below were done with endoscopic placement.

### Device Placement Under Anesthesia

All animal experiments were approved by and performed in accordance with the Committee on Animal Care at MIT. Yorkshire swine were obtained from Cummings School of Veterinary Medicine at Tufts University (Grafton, MA, USA) for in-vivo experiments. Pigs weighing approximately 60-100 kg were placed on a liquid diet for 24 hours the day before the study and were fasted overnight prior to the study. For device placement, animals were anesthetized using an intramuscular injection of midazolam 0.25 mg/kg and dexmedetomidine 0.03 mg/kg. Following sedation, animals were placed on thermal support and ophthalmic ointment was applied to both eyes. The animal was intubated and placed on isoflurane (2%) in oxygen and connected to a vital signs monitoring system. MiGUT was delivered orally into the stomach, using an orogastric tube and imaged using PENTAX EC-3870TLK (160 cm) to visualize the stomach with the animal in the left lateral position.

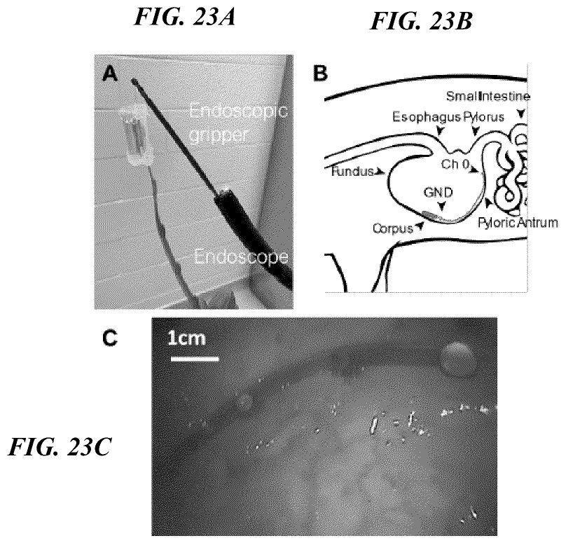

During these studies, two formfactors were tested. Initially, the device was placed with electrodes trailing the capsule as shown in FIG. 23A. To ensure no stress was placed on the capsule-electrode interface, a particularly brittle area after sealed with the epoxy, the device is pulled down the endoscope using the gripper. This ensured the electrodes were not damaged during delivery. Later, to showcase this could be taken orally without endoscopy, the roller configuration described elsewhere herein was used. This allowed the device to be pushed on the other side while protecting the electrode interface.

Nine experiments were performed under anesthesia, the data from which is shown below. In these experiments, the electrodes were positioned such that the reference electrode was localized near the stomach corpus as shown in FIGS. 23B-23C. These experiments lasted on average 1.5 hours.

### Data Processing

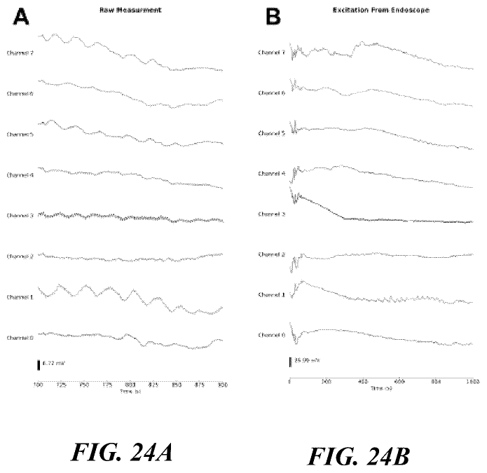

Biopotentials were recorded at 62.5 samples per second, with a representative sample shown in FIG. 24A. It is encouraging that a signal with similar to the slow wave can be easily picked out from most channels. Immediately following device placement (t=0 to 500 seconds), electrical activity is seen in all channels likely as a result of mechanical stimulation because of endoscopic visualization following device deployment (FIG. 24B). As such, only recordings approximately 10 min after the endoscope was removed considered with early noisy data being truncated. To isolate the multiple types of periodic waveforms observed, bandpass filtering using a third order Butterworth filter was used. In these experiments with the reference closest to channel zero, less of a change for channels closer to the reference was observed. Of the nine studies, one dataset was omitted due to a simultaneous study resulting in high levels of noise.

### Multimodal Signal Analysis

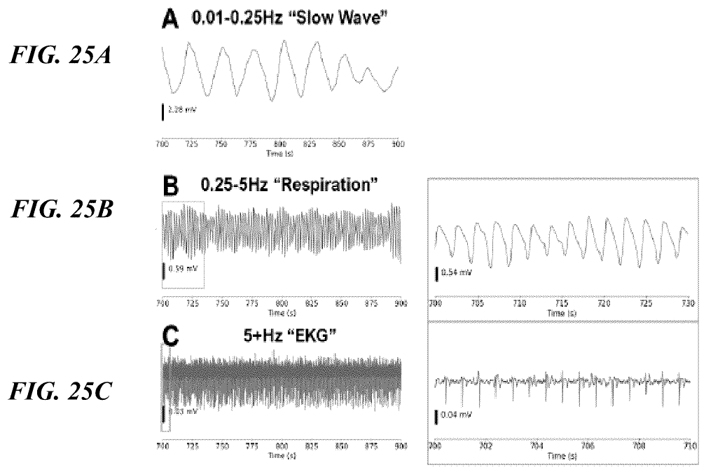

Three signals were seen to be superimposed on the raw signal shown in FIGS. 24A-24B. When filtering in the frequency of the slow wave, a continuous periodic waveform with mean amplitude of ˜5 mV and frequency approximately 3 cpm is seen (FIG. 25A). Similarly, filtering at a slightly higher frequency shows a periodic waveform with mean amplitude of ˜1 mV at a frequency of 26 cpm (FIG. 25B). Finally, at a frequency of 90 cpm a spiking waveform can be seen with mean amplitude of ˜0.05 mV (FIG. 25C). These components of high spectral energy correspond well with the recorded heart rate and respiratory rate monitored during the study. This supports that MiGUT can be used not just for tracking gastric slow waves, but also other vital signals which can be tracked individually or as an input to on-device algorithms.

In addition to the data presented in FIG. 24, an even slower periodic signal can be seen highlighted in FIG. 7B. Large amplitude waveforms were observed for six cycles with a period of approximately 500 seconds near the pylorus (chan. 0-2). This has not been previously reported in literature, possibly due to signal processing schemes focused on the expected slow wave frequency range. These signals may be attributed to the Migrating Motor Complex (MMC), which occurs on timescales from 90 to 120 minutes. These signals indicate additional control related activity either related to the MMC or as an intermediary process.

To ensure these signals are indeed of biological origin, in one study a barbiturate cocktail [Fatal Plus (sodium pentobarbital), 1 ml/10 lbs] was administered during a recording session. As seen in FIG. 7D, after administering the cocktail the slow wave (and all other signals) terminated. This indicates that these signals were not due to noise in the environment or the recording setup.

### Prokinetic Study

Finally, to show this system can track changes in motility, Azithromycin was administered halfway during six studies. Azithromycin is a prokinetic that has been shown to increases motilin receptors. It was intravenously delivered at 1 mg/kg over 5 minutes through an ear catheter. After administration, a significant increase in the amplitude of the slow wave was observed in five of the recording channels shown in FIG. 10A. However, no significant changes were seen in the respiration and heart rate frequency bands, with the EKG signal still clearly seen (FIG. 10B). This indicates throughout this duration of increased gastric activity (with suspected contractions), the electrodes continued to make good contact. Thus, MiGUT is shown to be able to track changes in gastric motility and maintain a reliable interface with the mucosa.

### In-Vivo Demonstration of MiGUT in an Ambulating Animal

After MiGUT was shown to reliably track multiple biopotentials in the stomach and maintain a quality connection with the mucosa, it is desirable to capture data from an ambulating large animal—something that has not been reported in an ingestible form factor.

Placement with Retention

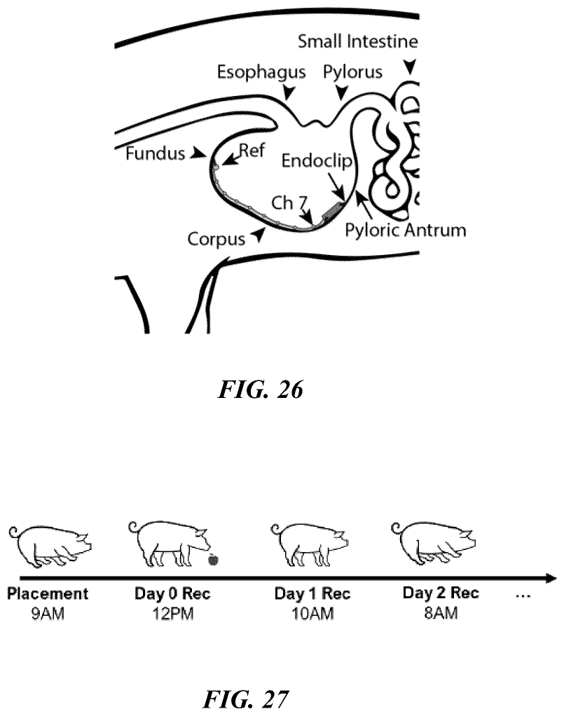

To ensure the device would not pass through the pylorus and have a better reference (with low electrical activity) over long recordings, the device was localized such that the reference was near the fundus with the electrodes running along the greater curvature of the stomach as shown in FIG. 26. Placement of the device mirrored what is described elsewhere herein with the exception that after delivery to the stomach, three endoclips (Boston Scientific Resolution Clips) were used to secure the device to the mucosa. These Resolution clips have been shown in literature to stay fixed for a few weeks, however much stronger endoclips such as the Ovesco clip could be used for longer residency up to 24 months. Two clips were used to secure the capsule on either side using nylon thread (FIG. 6), and an additional clip was used to secure the reference electrode by a nitinol wire loop through a hole on the reference electrode (FIG. 6).

### Study Design and Device Reliability

After placement (in the mornings), the device would sleep until approximately noon and record for 45 minutes. The device was then programmed to sleep for 22 hours to capture recordings during different activities as outlined in FIG. 27.

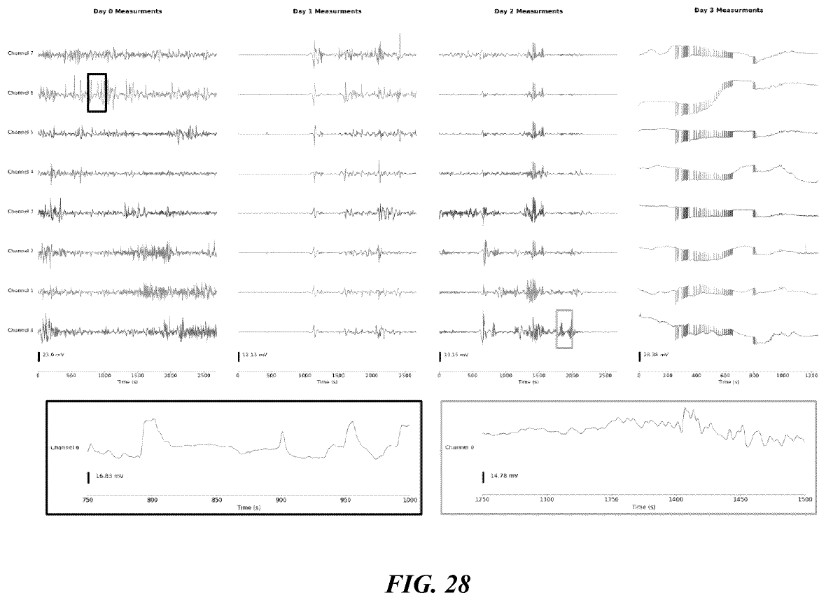

Over the course of four days, the quality of the signal was observed to be consistent and of high quality (after which the device ran out of battery) as shown in FIG. 28. While there are changes in signal amplitude, these are believed to be caused by changes in behavior described elsewhere herein and not due to degradation of the electrodes or device. Additionally, no motion artifacts are observed during these long recording sessions. Select areas of high activity are shown in FIG. 28 and can be seen to be smooth continuous changes in potential, and not similar to motion artifacts have been reported. This is transformational as motion artifacts have been a key limiting factor prevention adoption of gastric mapping systems, with signal processing algorithms developed specifically because of this to extract the underlying gastric signals.

This shows that MiGUT can be used reliably over multiple days, without any restrictions to the subjects' activities or diet. During the study, the pig was fed a combination of pellets, fruits, and vegetables, which did not interfere with any electrodes (all channels were able to obtain high quality measurements). Additionally, the peristaltic waves in the stomach did not dislodge the device. This showcases how MiGUT can be used for long term high quality recordings.

Fourteen studies were conducted, with one removed due to a poor ground connection (retention broke), and one the electrode broke during placement (large reference torn off in over tube). Different configurations were programmed to provide segmented data over multiple days as well as single continuous recordings for ˜3 hours. In eight of the successful studies, a dominant frequency of 4.39 cpm (SD: 0.27 cpm) is observed.

### Behavior Analysis

In six studies, 4.5 hours of video recordings were also taken simultaneously during recording sessions. At first, a GoPro (CA, USA; 1080p 30 fps) was used but later studies used mounted 1080p HD IP cameras with night vision to facilitate recording without human influence present. This video was first labeled, with each frame of video associated with a behavior: feeding, ambulating, sleeping, or playing. Then, the timestamps were synchronized with the recorded electrophysiological signals from MiGUT. FIGS. 10B-10D shows distinct changes in amplitude during different behaviors. Specifically the amplitude can be seen to be 3-6× larger when feeding compared to ambulation of resting, which is also seen in the frequency spectrum as a ˜3.2× increase in power.

### Passage Study

To validate this device could be safely implemented clinically, a passage study was conducted to ensure the device size (with long flexible electrodes) would not cause an obstruction after measurements were obtained. For tracking purposes, a device without the electronics was filled with 3.5-4 g of stainless steel powder (Sigma-Aldrich) matching the weight of MiGUT while ensuring clear visibility in X-Ray images. The largest electrode sizes, which had the thickest width along the ribbon, were used in these studies—smaller electrodes are hypothesized to pass easier. The modified device is shown in FIG. 5.

Over the course of three studies, all devices were shown to unroll in the stomach, passed into the intestines by day two, and were found excreted by day five (FIG. 5). One device was found to have the electrode torn off, which is believed to have occurred after excretion. This suggests the device can pass without obstruction and does not pose a safety risk. Further disassembly methods or dissolvable electrodes may be worth investigating for patient comfort.

### CONCLUSION

MiGUT is able to record high quality electrical signals from the gastric environment, including the gastric slow wave, heart rate, respiration rate, and other signals which require further study (possibly related to control of the migrating motor complex). Currently, this is believed to be the first ingestible device (in a swallowable formfactor) able to record from the mucosa over long periods of time, demonstrated in an awake, unrestricted pig. The results generally match those reported in literature, however a slightly higher slow wave frequency (4.39 cpm, N=8 ambulatory) compared to that reported in weaner pigs (3.2 cpm) and humans (2.8 cpm), but lower than canine (5.2 cpm) is described herein. In previous studies, this would be considered tachygastria, however as these results were consistent for long periods (>45 min), and thus it is believed this increase in frequency could either be attributed with ingestion of a bolus (which typically occurred approximately one hour before each recording) or due to lack of anesthesia. While not previously studied in context of the enteric nervous system, the fed motor pattern is characterized by contractions of irregular amplitude and frequency which could be promoted by a natural increase in the slow wave frequency. Similarly, as the migrating motor complex has been shown to decrease in frequency during sleep an interesting study would involve studying the impact of deep sleep on the slow wave. All previous high resolution studies have been done with the animal anesthetized which could have also impacted the slow wave frequency during their experiments. Opioids, which are commonly used in anesthesia, have previously been seen to significantly decrease or completely suppress the slow wave frequency and power in 11/19 patients. This may explain why the results from ambulating pigs are of higher frequency and higher amplitude than previously reported. MiGUT can facilitate more comprehensive studies to be conducted, continuously measuring before and during sedation.

MiGUT has also highlighted the variability in measurements that one might record from the stomach at different times. It has generally be considered that the slow wave is ever-present in the stomach, with superimposed rapid depolarizations from action potentials exceeding the threshold required to cause contractions. This has been reported in a number of studies spanning multiple animals in anesthetized and conscious settings. Interestingly, times where no slow wave activity was observed, in two separate animals for 45 minutes recording sessions coinciding with the animals being on liquid diet. In days prior and after, post feeding the slow wave was recorded with in the same animals with the devices in the same position. During these periods of quiescence the heart rate signal and respiratory signals can still be observed as in the anesthetized experiments, indicating this is not a device or contact issue. One possibility is that these recordings fell in phase I of the migrating motor complex, characterized by motor quiescence which can last up to 67 minutes in humans. Studies of the migrating motor complex have shown “Microwaves” (<9 mmHg) at the same frequency of the slow wave, but these were performed during invasive surgeries possibly causing additional stimulation. Longer term studies with MiGUT will provide more insight into electrical correlations with the migrating motor complex, fed motor pattern, and reveal the extent of these electrically quiescence periods.

Lastly, the optimal placement of the device and electrode configuration is still a topic of active investigation. The most interesting and impactful parameter varied was the location of the reference electrode. Acute, invasive systems have used a wide range of references far away from the stomach to provide a stable reference, such as the surface of the abdomen, hind leg, in tissue bath, while ambulating tethered systems have typically used bi-polar electrodes. MiGUT was positioned such that the reference was either placed in the corpus during anesthetized experiments or in the fundus during ambulating studies. Measurements taken referenced to the corpus were generally found to have a smaller variance over time compared to measurements referenced from the fundus, however other factors such as reference electrode clipping and different settings make it difficult to come to a clear conclusion. The fundus is believed to be the better choice as it naturally provides a stable, electrically quiet reference, but there may be an optimal distance from the measurement electrodes that would facilitate arbitrary placement (e.g., in the corpus) allowing for easier to implement electrode designs.

### Future Work

MiGUT opens many avenues of exploration for neural recordings in the gut. The interchangeable electrodes allows for further optimization and characterization of different electrode sizes and spacing, optimal reference location, and a platform to evaluate the quality of recordings from electrodes with different retention mechanisms. Specifically, replacing the endoclips used for retention with a mucoadhesive that provides a high quality electrical connection and secure each electrode for multiple days is advantageous, in some embodiments. In its current form, MiGUT facilitates a number of experiments to be done to better understand the enteric nervous system, slow wave, migrating motor complex, and fed motor pattern over long periods of time while not confined to a clinical setting. The size of the device would allow for multiple to be deployed at once if desired, allowing for simultaneous recordings throughout the GI tract. This could be especially useful, in some embodiments, when paired with disease models to better understand gastric neuropathies from an electrical perspective over long periods of time. Towards clinical translation, accommodation of the silver oxide batteries and further miniaturizing the size would be advantageous, which could be accomplished by using PCB manufacturing techniques or integrating all the components into a single custom integrated circuit, as known by those skilled in the art.

MiGUT can also be used as a general purpose, ultra small neural acquisition system to assist with other studies. As an example, MiGUT could be used in combination with Gastric Electric Stimulators to better understand what the optimal parameters are with high resolution recordings. Later, by adding stimulation capabilities to MiGUT, gastric stimulation could be provided in a closed loop manner for additional power savings and personalized therapy. Other avenues of parallel measurements include behavioral analysis, anesthesia monitoring/recovery, and other brain-gut axis studies.

Finally, ingestible electronics represent the forefront of biomedical devices that are small, power efficient, and can survive harsh environments—fundamental challenges that can be translated to a number of other biomedical and smart devices. There is still much work to be done in battery technology to increase the power density, low power communications which can effectively communicate with devices deep within the body, and durable device packaging that can survive the gastric environment for an ultra long period of time. Immediate steps to overcome these challenges in the present device, in some embodiments, include incorporating wireless charging or energy harvesting, custom antenna designs, and/or on-device processing to limit communications.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An article for monitoring electrical signals at a location internal to a subject, comprising:
a substrate comprising a plurality of electrodes, the article sized and adapted for ingestion and/or implantation at the location internal to the subject,
wherein the substrate has a Young's elastic modulus of greater than or equal to 0.01 MPa and less than or equal to 200 MPa, and wherein at the location internal to the subject, the substrate undergoes elastic recoil sufficient such that at least a portion of the plurality of electrodes contact a surface of a tissue at the location internal to the subject.

2. An article as in claim 1, further comprising a capsule, wherein the substrate is disposed within the capsule.

3. An article as in claim 1, wherein the plurality of electrodes is adjacent the substrate.

4. An article as in claim 1, wherein the substrate comprises a first configuration when disposed within the capsule.

5. An article as in claim 1, wherein, upon release from the capsule at the location internal to the subject, the substrate comprises a second configuration different from the first configuration.

6. An article as in claim 5, wherein the second configuration comprises at least a portion of the plurality of electrodes in contact with the surface of the tissue at the location internal to the subject.

7. An article as in claim 1, wherein the tissue is gastric tissue.

8. An article as in claim 1, wherein the location internal to the subject is the stomach of the subject.

9. An article as in claim 1, wherein the substrate has an aspect ratio of greater than or equal to 2.

10. An article as in claim 1, wherein the plurality of electrodes is in electrical communication with a processor.

11. An article as in claim 1, wherein an electrical signal determined by at least a portion of the plurality of electrodes is correlated with a gastric slow wave and/or gastric motility.

12. An article as in claim 1, wherein the first configuration is closed and the second configuration is open.

13. An article as in claim 1, wherein the substrate, when in its first configuration, is folded, rolled, bent, and/or compressed to fit within the capsule.

14. An article as in claim 1, wherein the substrate, when in its first configuration, is folded, rolled, bent, and/or compressed to facilitate internalization into the subject.

15. An article as in claim 1, wherein the article is an implantable article, further comprising endo-clips associated with the substrate.

16. An implantable article as in claim 15, wherein the endo-clips are configured to maintain a position of at least a portion of the implantable article at the location internal to the subject.

17. A method for monitoring electrical signals at a location internal to a subject using an article, comprising:
contacting a surface of a tissue with at least a portion of a plurality of electrodes of the article at the location internal to the subject; and
using the at least a portion of the plurality of electrodes to determine an electrical signal that is correlated with a gastric slow wave and/or gastric motility.

18. A method as in claim 17, further comprising wirelessly transmitting data from the location internal to the subject.

19. A method as in claim 18, wherein the data is associated with the electrical signal.

20. A method as in claim 18, wherein the data is received external the subject.

21-27. (canceled)
